(19) United States
(12) Patent Application Publication
(10) Pub. No.: US 2026/0066041 A1
(43) Pub. Date: Mar. 5, 2026

(54) DETECTING MUTATIONAL SIGNATURES WITH K-MER-BASED PSEUDOALIGNMENT

(71) Applicant: California Institute of Technology, Pasadena, CA (US)

(72) Inventors: Joseph M. Rich, Pasadena, CA (US); Lior S. Pachter, Pasadena, CA (US); Laura Luebbert, Pasadena, CA (US); Delaney Sullivan, Pasadena, CA (US)

(21) Appl. No.: 19/317,823

(22) Filed: Sep. 3, 2025

Related U.S. Application Data
(60) Provisional application No. 63/689,959, filed on Sep. 3, 2024.

(51) Int. Cl. G16B 20/20; G16B 30/10

(52) U.S. Cl. CPC G16B 20/20; G16B 30/10

(57) ABSTRACT

Disclosed herein include systems, devices, methods, and computer readable media for reference-based mutation or variant detection. The detection can be based on an index of variant-containing reference sequences and k-mer-based psuedoalignment.

### RELATED APPLICATIONS

This application claims the benefit under 35 U.S.C. § 119 (e) of U.S. Provisional Patent Application Ser. No. 63/689,959, filed Sep. 3, 2024, the content of this related application is incorporated herein by reference in its entirety for all purposes.

### REFERENCE TO SEQUENCE LISTING

The present application is being filed along with a Sequence Listing in electronic format. The Sequence Listing is provided as a file entitled 30KJ-810014-US_SequanceListing, created Aug. 21, 2025, which is 5 kilobytes in size. The information in the electronic format of the Sequence Listing is incorporated herein by reference in its entirety.

### BACKGROUND

### Field

The present disclosure relates generally to the field of variant calling.

### Description of the Related Art

The identification of genetic variants from sequencing reads with respect to a reference genome (variant calling) is a key step in many bioinformatics workflows. Genetic variants offer critical insights into both evolutionary biology and disease mechanisms, with many common conditions, such as Alzheimer's disease, cardiovascular disease, and cancer-harboring variants associated with pathogenicity or susceptibility. There is a need for a variant calling method that identifies variants with high speed, accuracy, and technology versatility

### SUMMARY

Disclosed herein include embodiments of method for determining one or more variants. In some embodiments, a method for determining one or more variants (or one or more steps thereof) is under control of a processor (e.g., a hardware processor or a virtual processor). The method can include: receiving variant information of a plurality of variants and an associated reference. The method can include: generating a plurality of variant-containing reference sequences (VCRSs) from the variant information of the plurality of variants and an associated reference. The method can include: building an index of the plurality of VCRSs. The method can include: receiving a plurality of sequence reads obtained from a sample. The method can include: determining one or more variants present in the plurality of sequence reads using the index of the plurality of VCRSs.

Disclosed herein include embodiments of a method for determining one or more variants. In some embodiments, a method for determining one or more variants (or one or more steps thereof) is under control of a processor (e.g., a hardware processor or a virtual processor). The method can comprise: receiving variant information of a plurality of variants and an associated reference. The method can comprise: generating a plurality of variant-containing reference sequences (VCRSs) from the variant information of the plurality of variants and an associated reference. The method can comprise: receiving a plurality of sequence reads obtained from a sample. The method can comprise: determining one or more variants present in the plurality of sequence reads using the plurality of VCRSs. In some embodiments, the method can comprise: building an index of the plurality of VCRSs. Determining the one or more variants present in the plurality of sequence reads can comprise: determining the one or more variants present in the plurality of sequence reads using the index of the plurality of VCRSs

In some embodiments, receiving the variant information of a plurality of variants comprises: receiving the variant information of the plurality of variants from a database of variants, optionally wherein the database of variants comprises Catalog Of Somatic Mutations In Cancer (COSMIC). In some embodiments, the reference associated with the variant information of the plurality of variants is the reference from which the plurality of variants are annotated. In some embodiments, the reference comprises a reference genome or a reference transcriptome.

In some embodiments, a VCRS (or one or more VCRS or each VCRS) of the plurality of VCRSs comprises a variant region flanked by a plurality of nucleotides (w) flanking each side of the variant region. w can be an integer. w can be different in different implementations. For example, w can be, 10, 20, 30, 40, 47, 50, 60, 70, 80, 90, 100, or more. In some embodiments, w is strictly less than k. In some embodiments, a VCRS (or one or more VCRS or each VCRS) of the plurality of VCRSs comprises a variant region flanked by a first plurality of nucleotides (w1) flanking left side of the variant region and a second plurality of nucleotides (w2) flanking right side of the variant region. w1 can be an integer. w2 can be an integer. w1 and w2 can be different in different implementations. For example, w1 and/or w2 can be 10, 20, 30, 40, 47, 50, 60, 70, 80, 90, 100, or more. w1 can be bigger than w2. w1 can be smaller than w2. w1 and w2 can be identical. In some embodiments, w1 is strictly less than k. w2 is strictly less than k.

In some embodiments, the method comprises: shortening a sequence flank (such as 1, 2, 3, 4, 5, 6, 7, 8, 9, 10, 11, 12, 13, 14, 15, 16, 17, 18, 19, 20, or more, nucleotides) of one or more VCRSs of the plurality of VCRSs. In some embodiments, the method comprises: trimming one or more VCRSs of the plurality of VCRSs prior to building the index of the plurality of VCRSs. In some embodiments, the method comprises: comprising filtering one or more VCRSs of the plurality of VCRSs prior to building the index of the plurality of VCRSs.

In some embodiments, each VCRS (or a VCRS or one or more VCRSs) corresponds to a variant (e.g., a different variant) of the plurality of variants. In some embodiments, each k-mer (or a k-mer or one or more k-mers) of a VCRS of the plurality of plurality of VCRS comprises at least one variant nucleotide (such as 1, 2, 3, 4, 5, 6, 7, 8, 9, 10, 11, 12, 13, 14, 15, 16, 17, 18, 19, 20, or more) of the corresponding variant of the plurality of variants. k can be an integer. k can be different in different implementations. For example, k can be 10, 20, 30, 40, 50, 51, 60, 70, 80, 90, 100, or more.

In some embodiments, the plurality of sequence reads is obtained from DNA sequencing, wherein the plurality of sequence reads is obtained from RNA sequencing, wherein the plurality of sequence reads is obtained from bulk RNAseq, and/or wherein the plurality of sequence reads is obtained from single-cell RNAseq. In some embodiments, the sample comprises: a DNA sample, an RNA sample, a bulk RNA sample, a single-cell RNA sample, or a combination thereof. In some embodiments, the plurality of sequence reads comprises a plurality of paired-end sequence reads, and/or wherein the plurality of sequence reads comprises a plurality of single-end sequence reads. In some embodiments, the sample comprises one or more cells, one or more single cells, cell-free DNA (cfDNA), cell-free RNA (cfRNA), or a combination thereof.

In some embodiments, the method comprises: trimming the plurality of sequence reads to generate a plurality of trimmed sequence reads. Determining one or more variants present in the plurality of sequence reads can comprise: determining one or more variants present in the plurality of trimmed sequence reads using the index of the plurality of VCRSs. In some embodiments, the method comprises: filtering the plurality of sequence reads to generate a plurality of filtered sequence reads. Determining one or more variants present in the plurality of sequence reads can comprise: determining one or more variants present in the plurality of filtered sequence reads using the index of the plurality of VCRSs.

In some embodiments, determining the one or more variants present in the plurality of sequence reads comprises: generating a variant count matrix. The variant count matrix can comprise a cell x variant matrix or a sample x variant matrix. Determining the one or more variants present in the plurality of sequence reads can comprise: determining the one or more variants present in the plurality of sequence reads using the variant count matrix. In some embodiments, the method comprises: validating the variant count matrix to generate a validated variant count matrix. Determining the one or more variants present in the plurality of sequence reads can comprise: determining the one or more variants present in the plurality of sequence reads using the validated variant count matrix. In some embodiments, the method comprises: thresholding the variant count matrix to generate a thresholded variant count matrix. Determining the one or more variants present in the plurality of sequence reads can comprises determining the one or more variants present in the plurality of sequence reads using the thresholded variant count matrix.

In some embodiments, determining the one or more variants in the plurality of sequence reads comprises: determining the one or more variants present in the plurality of sequence reads using pseudoalignment (or quasialignment). The pseudoalignment can comprise k-mer-based pseudoalignment. The pseudoalignment can comprise kallisto pseudoalignment. In some embodiments, pseudoalignment comprises building a k-mer hash map. The pseudoalignment can comprise creating a de Bruijn graph.

Disclosed herein include embodiments of a system. The system can comprise: non-transitory memory configured to store executable instructions. The system can comprise: a hardware processor in communication with the non-transitory memory. The hardware processor can be programmed by the executable instructions to perform: any method of the present disclosure.

Disclosed herein include embodiments of a system for determining one or more variants. In some embodiments, a system for determining one or more variants comprises: non-transitory memory configured to store executable instructions. The non-transitory memory can be configured to store an index of a plurality of variant-containing reference sequences (VCRSs). The plurality of VCRSs can be generated from variant information of a plurality of variants and an associated reference. The system can include: a hardware processor in communication with the non-transitory memory. The hardware processor can be programmed by the executable instructions to perform: receiving a plurality of sequence reads obtained from a sample. The hardware processor can be programmed by the executable instructions to perform: determining one or more variants present in the plurality of sequence reads using the index of the plurality of VCRSs.

Disclosed herein include embodiments of a system for determining one or more variants. In some embodiments, a system for determining one or more variants comprises: non-transitory memory configured to store executable instructions. The non-transitory memory can be configured to store a plurality of variant-containing reference sequences (VCRSs). The plurality of VCRSs can be generated from variant information of a plurality of variants and an associated reference. The system can include a hardware processor in communication with the non-transitory memory. The hardware processor can be programmed by the executable instructions to perform: receiving a plurality of sequence reads obtained from a sample. The hardware processor can be programmed by the executable instructions to perform: determining one or more variants present in the plurality of sequence reads using the plurality of VCRSs. The hardware processor can be programmed by the executable instructions to perform: building an index of the plurality of VCRSs. Determining the one or more variants present in the plurality of sequence reads can comprise: determining the one or more variants present in the plurality of sequence reads using the index of the plurality of VCRSs.

In some embodiments, the variant information of the plurality of variants is received from a database of variants. The database of variants can comprise Catalog Of Somatic Mutations In Cancer (COSMIC). In some embodiments, the reference associated with the variant information of the plurality of variants is the reference from which the plurality of variants are annotated. In some embodiments, the reference comprises a reference genome or a reference transcriptome.

In some embodiments, a VCRS (or one or more VCRS or each VCRS) of the plurality of VCRSs comprises a variant region flanked by a plurality of nucleotides (w) flanking each side of the variant region. w can be an integer. w can be different in different implementations. For example, w can be, 10, 20, 30, 40, 47, 50, 60, 70, 80, 90, or 100. In some embodiments, w is strictly less than k. In some embodiments, a VCRS (or one or more VCRS or each VCRS) of the plurality of VCRSs comprises a variant region flanked by a first plurality of nucleotides (w1) flanking left side of the variant region and a second plurality of nucleotides (w2) flanking right side of the variant region. w1 can be an integer. w2 can be an integer. w1 and w2 can be different in different implementations. For example, w1 and/or w2 can be 10, 20, 30, 40, 47, 50, 60, 70, 80, 90, or 100. w1 can be bigger than w2. w1 can be smaller than w2. w1 and w2 can be identical. In some embodiments, w1 is strictly less than k. w2 is strictly less than k.

In some embodiments, a sequence flank of one or more VCRSs of the plurality of VCRSs is shortened (e.g., by 1, 2, 3, 4, 5, 6, 7, 8, 9, 10, 11, 12, 13, 14, 15, 16, 17, 18, 19, 20, or more, nucleotides). In some embodiments, one or more VCRSs of the plurality of VCRSs is trimmed and/or filtered.

In some embodiments, each VCRS (or a VCRS or one or more VCRSs) corresponds to a variant (e.g., a different variant) of the plurality of variants. In some embodiments, each k-mer (or a k-mer or one or more k-mers) of a VCRS of the plurality of plurality of VCRS comprises at least one variant nucleotide (such as 1, 2, 3, 4, 5, 6, 7, 8, 9, 10, 11, 12, 13, 14, 15, 16, 17, 18, 19, 20, or more) of the corresponding variant of the plurality of variants. k can be an integer. k can be different in different implementations. For example, k can be 10, 20, 30, 40, 50, 51, 60, 70, 80, 90, 100, or more.

In some embodiments, the plurality of sequence reads is obtained from DNA sequencing, wherein the plurality of sequence reads is obtained from RNA sequencing, wherein the plurality of sequence reads is obtained from bulk RNAseq, and/or wherein the plurality of sequence reads is obtained from single-cell RNAseq. In some embodiments, the sample comprises: a DNA sample, an RNA sample, a bulk RNA sample, a single-cell RNA sample, or a combination thereof. In some embodiments, the plurality of sequence reads comprises a plurality of paired-end sequence reads, and/or wherein the plurality of sequence reads comprises a plurality of single-end sequence reads. In some embodiments, the sample comprises one or more cells, one or more single cells, cell-free DNA (cfDNA), cell-free RNA (cfRNA), or a combination thereof.

In some embodiments, the hardware processor is programmed by the executable instructions to perform: trimming the plurality of sequence reads to generate a plurality of trimmed sequence reads. Determining one or more variants present in the plurality of sequence reads can comprise: determining one or more variants present in the plurality of trimmed sequence reads using the index of the plurality of VCRSs. In some embodiments, the hardware processor is programmed by the executable instructions to perform: filtering the plurality of sequence reads to generate a plurality of filtered sequence reads. Determining one or more variants present in the plurality of sequence reads can comprise: determining one or more variants present in the plurality of filtered sequence reads using the index of the plurality of VCRSs.

In some embodiments, determining the one or more variants present in the plurality of sequence reads comprises: generating a variant count matrix. The variant count matrix can comprise a cell x variant matrix or a sample x variant matrix. Determining the one or more variants present in the plurality of sequence reads can comprise: determining the one or more variants present in the plurality of sequence reads using the variant count matrix. In some embodiments, the hardware processor is programmed by the executable instructions to perform: validating the variant count matrix to generate a validated variant count matrix. Determining the one or more variants present in the plurality of sequence reads can comprise: determining the one or more variants present in the plurality of sequence reads using the validated variant count matrix. In some embodiments, the hardware processor is programmed by the executable instructions to perform: thresholding the variant count matrix to generate a thresholded variant count matrix. Determining the one or more variants present in the plurality of sequence reads can comprises determining the one or more variants present in the plurality of sequence reads using the thresholded variant count matrix.

In some embodiments, determining the one or more variants in the plurality of sequence reads comprises: determining the one or more variants present in the plurality of sequence reads using pseudoalignment (or quasialignment). The pseudoalignment can comprise k-mer-based pseudoalignment. The pseudoalignment can comprise kallisto pseudoalignment. In some embodiments, pseudoalignment comprises building a k-mer hash map. The pseudoalignment can comprise creating a de Bruijn graph.

Disclosed herein include embodiments of a computer readable medium. In some embodiments, a computer readable medium comprising executable instructions, when executed by a processor (e.g., a hardware processor or a virtual processor) of a computing system or a device, cause the processor, to perform any method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1A-FIG. 1K show a non-limiting exemplary overview of varseek and its applications to variant calling in single-cell RNA-sequencing data (scRNA-seq). FIG. 1A depicts a schematic of inputs and outputs of the standard varseek workflow. FIG. 1B shows a Principal Component Analysis (PCA) plot in gene expression space of healthy (blue) and neoplastic (orange) astrocytes from a glioblastoma dataset. FIG. 1C shows a variant scatterplot of fraction of cells with variant (FCV) in neoplastic vs. healthy astrocytes. FIG. 1D shows a gene scatterplot of log fold change (logFC) in gene expression between neoplastic and healthy astrocytes vs. the maximum FCV among cancer-specific variants for that gene. FIG. 1E shows a UpSet plot of cancer-specific variants as identified by scRNA-seq data compared to bulk RNA-seq data from the cancer cell line encyclopedia (CCLE). FIG. 1F shows a heatmap of FCV for the top 50 cancer-specific variants across the 12 cell types previously identified. FIG. 1G shows a volcano plot of gene expression between neoplastic cells with vs. without the C3:4989C>T variant detected. FIG. 1H shows a cell scatterplot of PCI loading value vs. total COSMIC mutations detected. FIG. 1I illustrates loadings of PC1, with some of the genes identified as cancer-specific by cBioPortal labeled. FIG. 1J show violin plots of total COSMIC mutations detected across all cells for each gene. Black points=values for the cell circled in black in FIG. 1H. FIG. 1K show lollipop plots displaying COSMIC mutations in four selected genes for the cell circled in black in FIG. 1H.

FIG. 2A-FIG. 2I illustrate the sensitivity of the varseek method in detecting variants. FIG. 2A-FIG. 2C: Recall vs. variant read depth for substitutions (FIG. 2A), deletions (FIG. 2B), and insertions (FIG. 2C). FIG. 2D-FIG. 2F: Recall vs. VAF for substitutions (FIG. 2D), deletions (FIG. 2E), and insertions (FIG. 2F) at a fixed variant read depth of 32. FIG. 2G is a Principal Component Analysis (PCA) plot in variant space of African ancestry (blue) and European ancestry (orange) of samples from the Geuvadis dataset. FIG. 2H is a variant detection histogram corresponding of samples from the Geuvadis dataset. FIG. 2I illustrates Superpopulation Silhouette Score (blue) and Recall at downsampled fraction (orange) across multiple downsampled fractions of data.

FIG. 3A-FIG. 3F illustrate the varseek approach is fast and memory-efficient. FIG. 3A-FIG. 3B: Variant calling runtime (FIG. 3A) and maximum memory usage (FIG. 3B) of varseek and multiple other variant calling tools by number of reads in the sequencing data. FIG. 3C-FIG. 3D: vk ref runtime (FIG. 3C) and maximum memory usage (FIG. 3D) by number of entries in the variant database. FIG. 3E-FIG. 3F: vk count runtime (FIG. 3E) and maximum memory usage (FIG. 3F) by number of entries in the variant database.

FIG. 4 is a non-limiting exemplary schematic of varseek. vk ref wraps vk build, vk info, vk filter, and kb ref. vk count wraps vk fastqpp, kb count, vk clean, and vk summarize. Example file representations of major inputs/outputs are shown below their position in the pipeline. Solid arrows show required inputs/outputs in some embodiments; dashed arrows show optional inputs/outputs in some embodiments.

FIG. 5 depicts a non-limiting exemplary variant reference index file creation process by vk ref from the COSMIC CMC database for analysis of Glioblastoma datasets.

FIG. 6A-FIG. 6F: Glioblastoma analysis between single-cell RNA-seq (scRNA-seq) data and Cancer Cell Line Encyclopedia (CCLE) bulk RNA-seq data. FIG. 6A: Histogram of fraction of samples with variant (FSV) for all variants in CCLE data. FSV=0.5 used as a threshold for denoting a cancer-specific variant in CCLE data (dashed gray line). FIG. 6B: UpSet plot comparing the sets of cancer-specific genes as identified by cBioPortal TCGA (mutated in at least 10 samples), scRNA-seq data (containing a mutation that is present in at least 2.5% of neoplastic cells and in a higher percentage of neoplastic cells than healthy cells), and CCLE (FSV≥0.5). FIG. 6C-FIG. 6D: Scatterplot for variants of scRNA-seq neoplastic fraction of cells with variant (FCV) vs. CCLE FSV. FIG. 6C is for all variants, and FIG. 6D is for cancer-specific variants (cBio definition). FIG. 6E-FIG. 6F: Scatterplot for genes of scRNA-seq neoplastic cells max FCV across all variants for the gene vs. CCLE max FSV across all variants for the gene. FIG. 6E is for all genes, and FIG. 6F is for genes of cancer-specific variants (cBio definition).

FIG. 7A-FIG. 7M illustrate creation of synthetic RNA-seq dataset and analysis of variant detection. FIG. 7A is a non-limiting exemplary schematic of the inputs and outputs to the function vk sim to produce the dataset. Each combination of conditions in # variant-containing reads, # wt reads, and variant info had 350 variants drawn from it. FIG. 7B-FIG. 7D: UpSet plot of detection of substitution (FIG. 7B), deletion (FIG. 7C), and insertion (FIG. 7D) Variants with a minimum variant read depth of 3 across all tools. FIG. 7E-FIG. 7M: Analysis of indels with a minimum variant read depth of 128, stratified by those detected by varseek and at most one other tool (orange) or varseek and at least two other tools (blue). FIG. 7E: Fraction of variants in each variant type. FIG. 7F: Average number of variants per gene. FIG. 7G: Fraction of variants that disrupt a homopolymer (at least three identical bases in a row). FIG. 7H-FIG. 7J: Variant length for deletions (FIG. 7H), insertions (FIG. 7I), and duplications (FIG. 7J). FIG. 7K-FIG. 7M: Local sequence complexity (defined by the number of unique triplets in a reading frame within 15 nucleotides of the variant) for deletions (FIG. 7K), insertions (FIG. 7L), and duplications (FIG. 7M).

FIG. 8A-FIG. 8C depict non-limiting exemplary visualization of the pileups of three variants detected by only varseek and at most one other tool. FIG. 8A: ENST00000329758:c.677del (a deletion). FIG. 8B: ENST00000300305:c.975_977dup (a duplication). FIG. 8C: ENST00000372991:c.995 996insTCCAGCCCAGC (an insertion). Note that positions are given relative to the cDNA of each transcript.

FIG. 9A-FIG. 9E illustrate validation of varseek on a sample from the Geuvadis dataset. FIG. 9A is an UpSet plot comparing variants detected by varseek in bulk RNA-seq to variants detected from GATK HaplotypeCaller in gene-coding regions of whole genome sequencing. FIG. 9B is an UpSet plot similar to FIG. 9A but with the addition of an entry for varseek applied to whole exome sequencing. FIG. 9C: Explanation for cases where varseek did not detect a read and GATK HaplotypeCaller did detect a read. FIG. 9D: Explanation for cases where varseek detected a read and GATK HaplotypeCaller did not detect a read. FIG. 9E: Stratification by number of reads of variants undetected by varseek but detected by both GATK HaplotypeCaller and pileup.

FIG. 10A-FIG. 10H depict non-limiting exemplary PCA plots of Geuvadis data colored by superpopulation at different downsampled fractions corresponding to FIG. 2I. Each percent provides the amount of data remaining (i.e., 1—percentage downsampled). (FIG. 10A) 1%, (FIG. 10B) 2%, (FIG. 10C) 4%, (FIG. 10D) 8%, (FIG. 10E) 16%, (FIG. 10F) 32%, (FIG. 10G) 64%, and (FIG. 10H) 100% (full data).

FIG. 11A-FIG. 11H depict non-limiting exemplary variant detection histograms corresponding to FIG. 2I. Each percent provides the amount of data remaining (i.e., 1-percentage downsampled). (FIG. 11A) 1%, (FIG. 11B) 2%, (FIG. 11C) 4%, (FIG. 11D) 8%, (FIG. 11E) 16%, (FIG. 11F) 32%, (FIG. 11G) 64%, and (FIG. 11H) 100% (full data).

FIG. 12A-FIG. 12F illustrate non-limiting exemplary methods implemented in varseek for controlling for false positives. All solutions are described in more detail in the Discussion and Methods sections. FIG. 12A: Case when a k-mer from the variant containing reference sequence (VCRS) is shared with a k-mer in the non-variant containing sequence. FIG. 12B: Case when a k-mer from the VCRS is shared with a k-mer from another part of the reference genome or transcriptome. FIG. 12C: Case when a k-mer from the VCRS is shared with a nearby variant. FIG. 12D: Case when a low-quality read region erroneously contains a VCRS k-mer due to sequencing error. FIG. 12E: Case when a k-mer from the VCRS is shared with a distant variant. FIG. 12F: Case when a high-quality read region erroneously contains a VCRS k-mer due to sequencing error.

FIG. 13 is a non-limiting exemplary process overview for detecting mutational signatures with k-mer-based psuedoalignment. The first step involved the creation of an optimized MCRS index from a reference genome and a mutation database. The software tools gget mutate and kb ref were specifically designed and expanded to implement this first step. Additionally, this step involved acquiring DNA or RNA data from a patient or sample (before or after index generation). The second step involved the alignment of DNA/RNAseq reads to the optimized MCRS index to create a sample/cell by mutation count matrix. This second step was specifically implemented with the software package kb count. kb, kallisto-bustools; MCRS, mutation-containing reference sequence.

FIG. 14 is a non-limiting exemplary illustration of controlling for false positives. The process to control for false positives involved (i) quality control and MCRS flank length adjustment using gget mutate, (ii) true alignment of each k-mer from each MCRS entry to the reference genome and transcriptome with bowtie2, with subsequent removal of any MCRS entry containing a k-mer that aligns anywhere in the reference transcriptome, and (iii) pseudoalignment of each MCRS entry to the reference transcriptome with kb, with subsequent removal of any MCRS entry that aligns to the reference transcriptome. kb, kallisto-bustools.

FIG. 15A-FIG. 15B show data of detecting mutational signatures with k-mer-based psuedoalignment. FIG. 15A: The top-mutated genes identified by the workflow disclosed herein from an esophageal cancer cell line. The identified mutated genes matched with mutations or gene alterations previously associated with esophageal cancer (indicated by red box). FIG. 15B: By investigating the distribution of gene-altering events across several patients suffering from the same disease, the frequency with which each gene was altered was identified.

FIG. 16 is a block diagram of an illustrative computing system that can be used in some embodiments to execute the processes and implement the features described herein, for example, variant detection.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include embodiments of method for determining one or more variants. In some embodiments, a method for determining one or more variants (or one or more steps thereof) is under control of a processor (e.g., a hardware processor or a virtual processor). The method can include: receiving variant information of a plurality of variants and an associated reference. The method can include: generating a plurality of variant-containing reference sequences (VCRSs) from the variant information of the plurality of variants and an associated reference. The method can include: building an index of the plurality of VCRSs. The method can include: receiving a plurality of sequence reads obtained from a sample. The method can include: determining one or more variants present in the plurality of sequence reads using the index of the plurality of VCRSs.

Disclosed herein include embodiments of a method for determining one or more variants. In some embodiments, a method for determining one or more variants (or one or more steps thereof) is under control of a processor (e.g., a hardware processor or a virtual processor). The method can comprise: receiving variant information of a plurality of variants and an associated reference. The method can comprise: generating a plurality of variant-containing reference sequences (VCRSs) from the variant information of the plurality of variants and an associated reference. The method can comprise: receiving a plurality of sequence reads obtained from a sample. The method can comprise: determining one or more variants present in the plurality of sequence reads using the plurality of VCRSs. In some embodiments, the method can comprise: building an index of the plurality of VCRSs. Determining the one or more variants present in the plurality of sequence reads can comprise: determining the one or more variants present in the plurality of sequence reads using the index of the plurality of VCRSs.

Disclosed herein include embodiments of a system. The system can comprise: non-transitory memory configured to store executable instructions. The system can comprise: a hardware processor in communication with the non-transitory memory. The hardware processor can be programmed by the executable instructions to perform: any method of the present disclosure.

Disclosed herein include embodiments of a system for determining one or more variants. In some embodiments, a system for determining one or more variants comprises: non-transitory memory configured to store executable instructions. The non-transitory memory can be configured to store an index of a plurality of variant-containing reference sequences (VCRSs). The plurality of VCRSs can be generated from variant information of a plurality of variants and an associated reference. The system can include: a hardware processor in communication with the non-transitory memory. The hardware processor can be programmed by the executable instructions to perform: receiving a plurality of sequence reads obtained from a sample. The hardware processor can be programmed by the executable instructions to perform: determining one or more variants present in the plurality of sequence reads using the index of the plurality of VCRSs.

Disclosed herein include embodiments of a system for determining one or more variants. In some embodiments, a system for determining one or more variants comprises: non-transitory memory configured to store executable instructions. The non-transitory memory can be configured to store a plurality of variant-containing reference sequences (VCRSs). The plurality of VCRSs can be generated from variant information of a plurality of variants and an associated reference. The system can include a hardware processor in communication with the non-transitory memory. The hardware processor can be programmed by the executable instructions to perform: receiving a plurality of sequence reads obtained from a sample. The hardware processor can be programmed by the executable instructions to perform: determining one or more variants present in the plurality of sequence reads using the plurality of VCRSs. The hardware processor can be programmed by the executable instructions to perform: building an index of the plurality of VCRSs. Determining the one or more variants present in the plurality of sequence reads can comprise: determining the one or more variants present in the plurality of sequence reads using the index of the plurality of VCRSs.

Disclosed herein include embodiments of a computer readable medium. In some embodiments, a computer readable medium comprising executable instructions, when executed by a processor (e.g., a hardware processor or a virtual processor) of a computing system or a device, cause the processor, to perform any method disclosed herein.

### Definitions

Unless defined otherwise, technical and scientific terms used herein have the same meaning as commonly understood by one of ordinary skill in the art to which the present disclosure belongs.

Reference-Based Variant Detection with Varseek

Variant detection from sequencing data is fundamental for genomics and is the first step in a wide range of applications, ranging from genome-wide association studies to disease diagnosis. Widely used tools for variant detection utilize a de novo approach that is based on a combination of read mapping algorithms and statistical methods for identifying genetic variation from error-prone sequencing data. This approach has been successful, although the detection of insertion and deletion variants, as well as the detection of variants from low-coverage data, remain challenging problems. Disclosed herein is a reference-based approach (referred to as varseek) to variant detection that improved performance in these challenging cases. The varseek approach utilizes a k-mer pseudoalignment approach, which provides the ability to identify variants at single-cell resolution in single-cell transcriptomics data. The versatility and performance of varseek for detecting tumor-specific COSMIC variants in glioblastoma single-cell sequencing is shown.

The identification of genetic variants from sequencing reads with respect to a reference genome (variant calling) is a key step in many bioinformatics workflows. Traditional variant calling involves aligning sequencing reads to a reference genome, followed by quality control and statistical testing to determine positions in the reference genome where a collection of reads can be ascertained to differ from the reference genome. Genetic variants offer critical insights into both evolutionary biology and disease mechanisms, with many common conditions, such as Alzheimer's disease, cardiovascular disease, and cancer-harboring variants associated with pathogenicity or susceptibility.

Cancer diagnostics and treatment, in particular, is an important application of variant calling, as there may be heterogeneous factors driving the disease that are unique to each individual and depend on specific mutations. The ability to efficiently describe the mutations driving cancer in an individual is crucial for precision medicine and can enable targeted therapies explicitly tailored to each patient's disease. Determining cancer-driven and cancer-driving mutations in a patient, which can be at single-cell resolution, is challenging. It requires characterizing the millions of cancer-associated mutations, identifying the mutations present in a sample derived from a patient, and distinguishing relevant mutations from technical noise or noncausal biological variation while tracking the cell of origin of mutations.

Several databases have curated large-scale annotated lists of cancer-associated mutations. The Catalogue of Somatic Mutations in Cancer (COSMIC) is one such database, cataloging almost 24 million cancer-associated somatic genomic variants. COSMIC consists of multiple projects, including the cancer mutation census (CMC), whole and targeted genome screens, the cancer gene census, drug resistance screens, and cell lines. Each project contains cancer mutations derived from different sources or filtered by varying levels of importance. One group identified 853 pathogenic or likely pathogenic variants from 10,389 cases across 33 cancer types in The Cancer Genome Atlas Program (TCGA). In addition, the Memorial Sloan Kettering Integrated Mutation Profiling of Actionable Cancer Targets (MSK-IMPACT) project has documented 78,066 cancer mutations in 341 key cancer genes. The IntOGen meta-database has compiled 257,898,749 mutations across several mutation databases, including TCGA, cBioPortal, and the International Cancer Genome Consortium (ICGC).

Current de novo variant detection approaches do not take advantage of these variant databases to improve the sensitivity and interpretability of the variant calling process. Additionally, some existing variant calling tools require significant runtime, on the order of hours to days, for standard sequencing results, and are unable to analyze data from single-cell technologies.

In addition to the creation of variant databases, there has been a steady increase in the amount of genomic sequencing data produced. Approximately 1021 bases are sequenced each year, generating 1 million TB of data annually. The rate of research studies including next-generation sequencing data is growing rapidly, including studies involving single-cell resolution. Sequencing becomes cheaper each year, with the cost of sequencing a human genome currently costing less than $1,000. Sequencing data is becoming more cost-effective and widespread in a clinical setting, with Medicare cost estimates between $500 and $1000 for targeted sequencing assays and $1499.32-3388.18 for whole exome sequencing. The increasing adoption of sequencing in the clinic motivates increased accuracy and speed of variant calling in order to allow for real-time diagnosis and treatment planning.

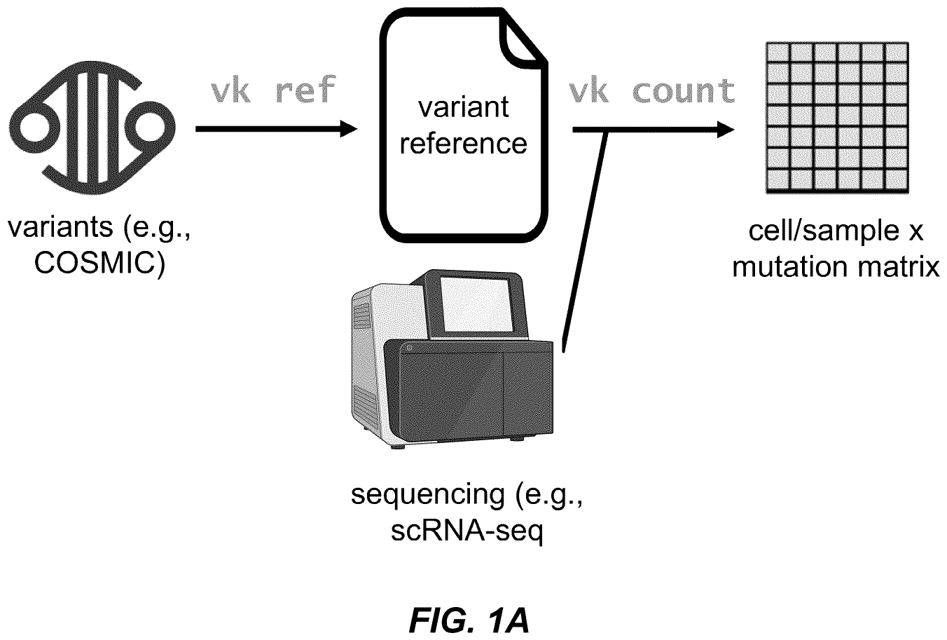

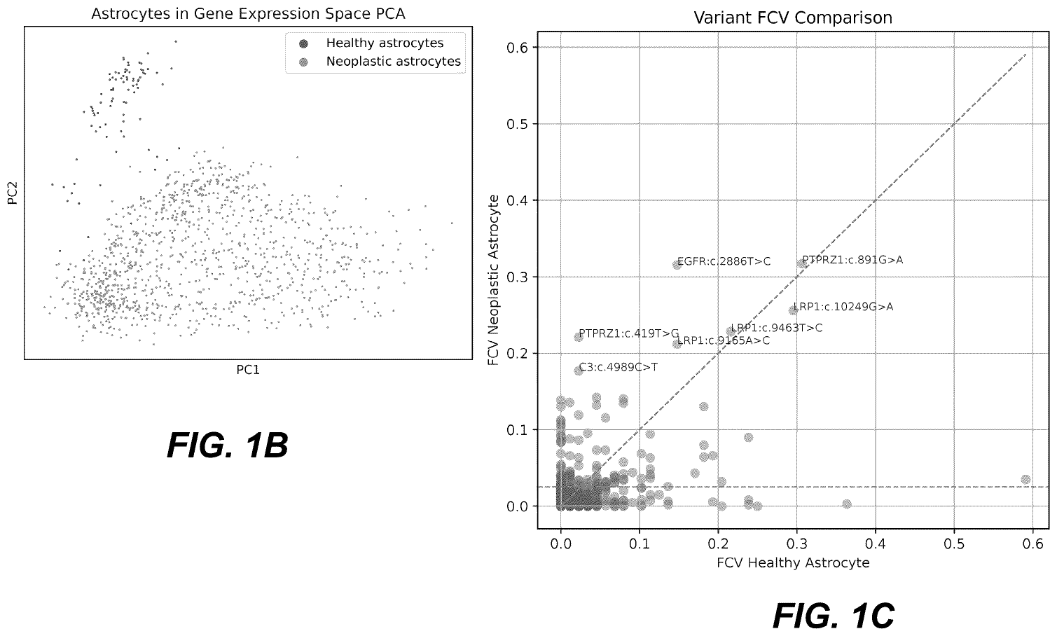

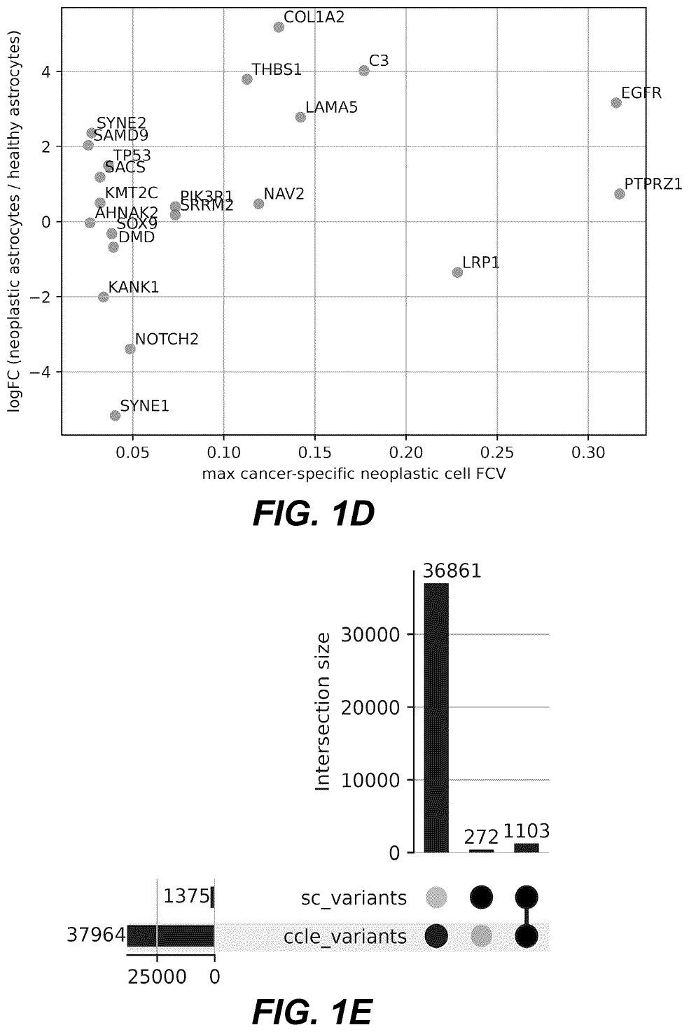

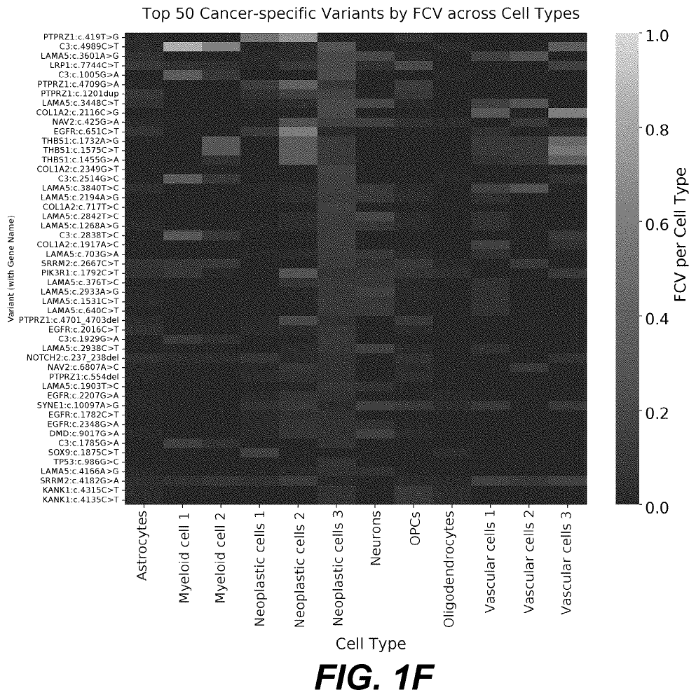

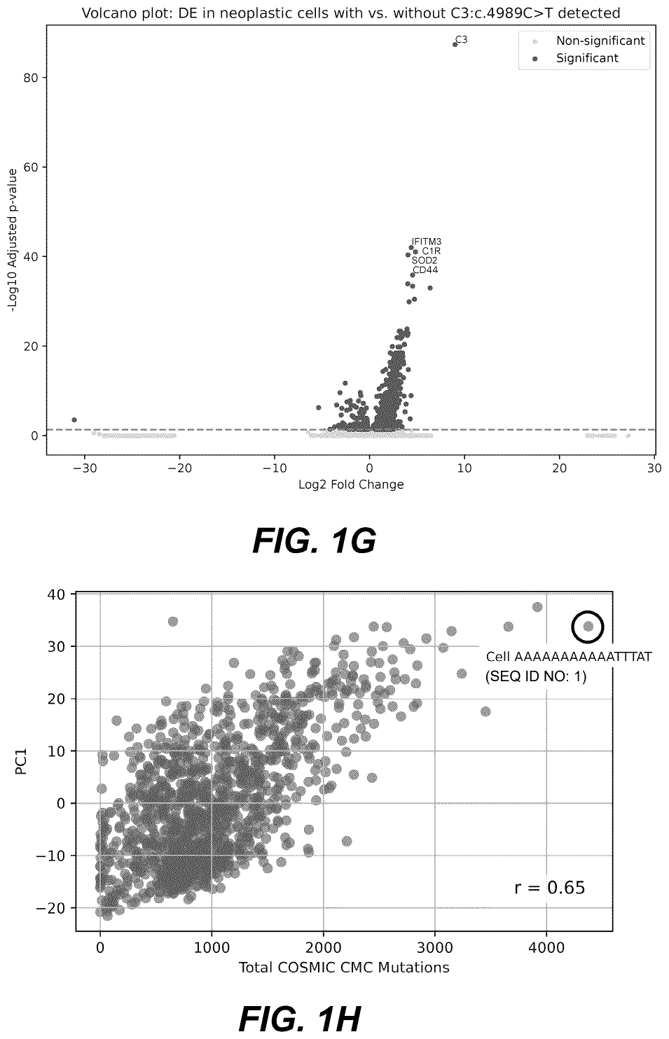

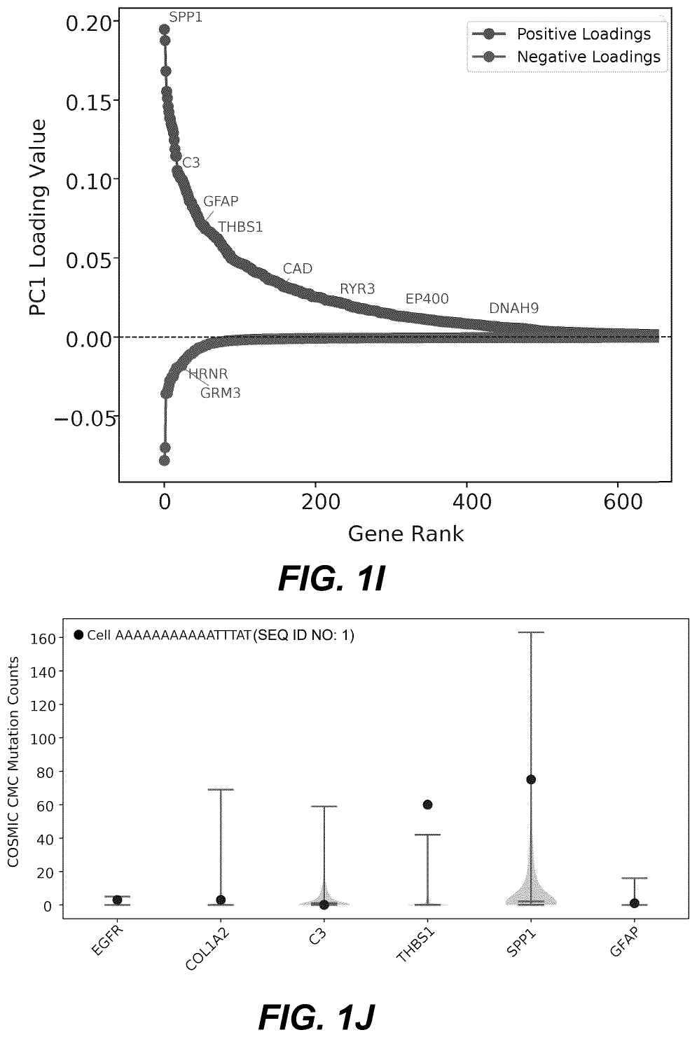

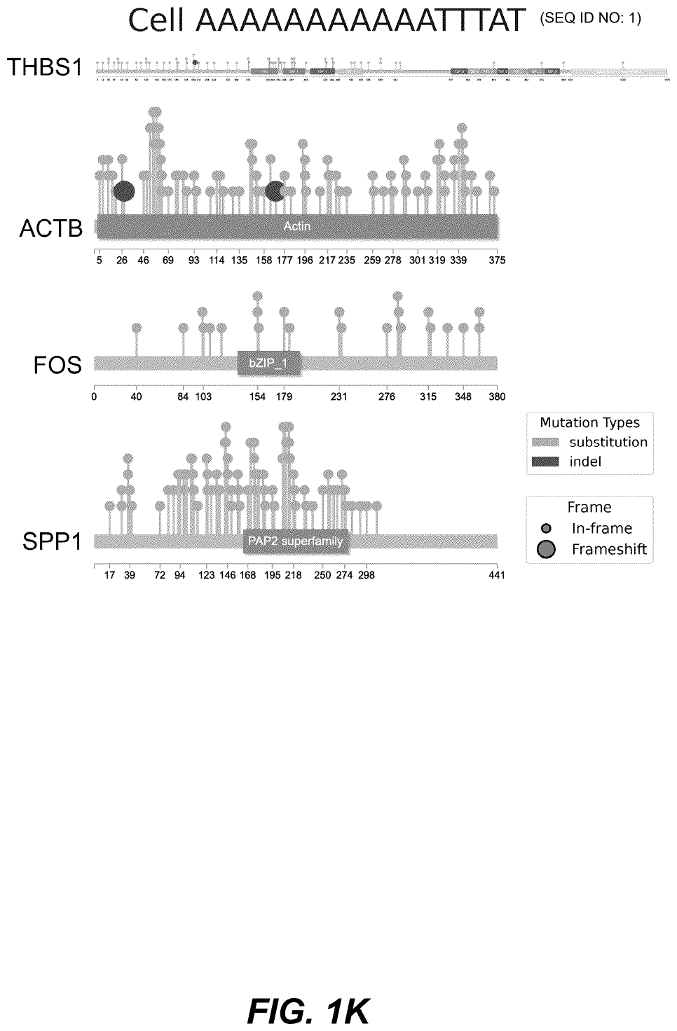

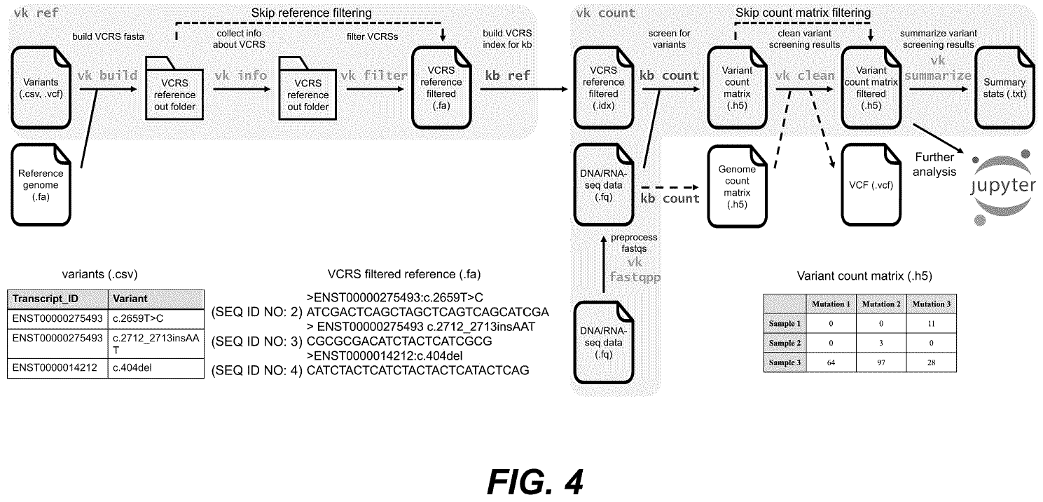

Disclosed herein is a reference-based variant calling tool (referred to as varseek) that identifies variants with high speed, accuracy, and technology versatility. The varseek program takes as input a reference database of variants and sequencing data from any sequencing technology spanning DNA, bulk RNA, and single-cell RNA sequencing (see Method section, FIG. 1A and FIG. 4). The workflow underlying varseek can involve the creation of a reference of variant-containing sequences and the identification of variants to this reference via k-mer matching using kallisto followed by filtering. The use of varseek in to the analysis of cancer single-cell heterogeneity and population analysis across DNA, bulk RNA, and single-cell RNA (scRNA) sequencing is described herein.

### Results

### Varseek Identified Mutations at Single-Cell Resolution

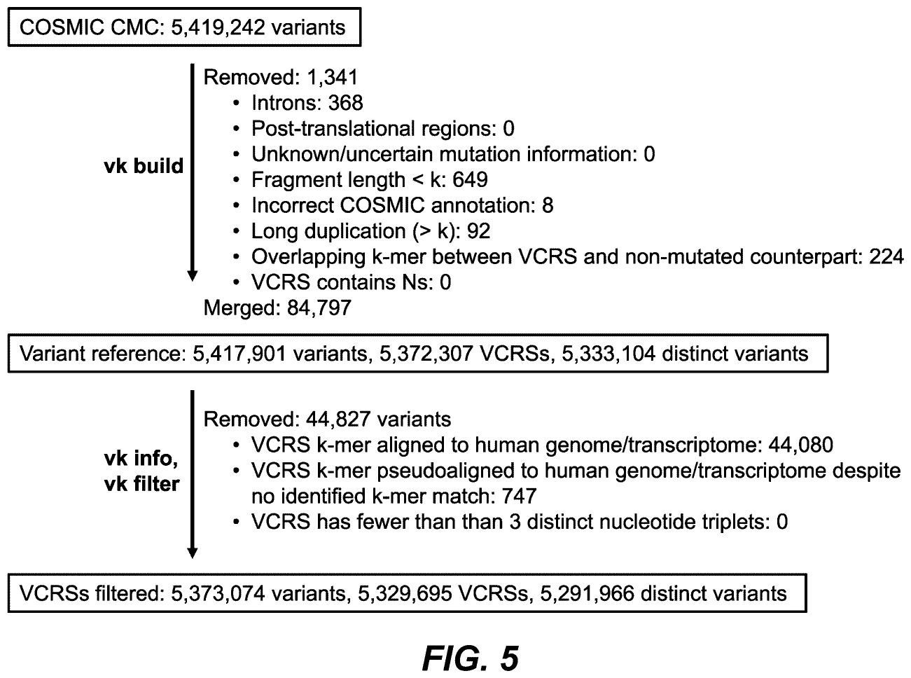

To illustrate the ability of varseek to identify mutations at single-cell resolution, variant calling was performed with varseek on scRNA-seq glioblastoma multiforme (GBM) data generated by Smart-Seq v2 using a variant index generated from the COSMIC CMC database, which catalogs 5,419,242 mutations associated with cancer (FIG. 5). The neoplastic and healthy astrocytes were separated in the 2D principal component analysis (PCA) projection based on gene expression (FIG. 1B). Cancer-specific variants were defined as those present in at least 2.5% of the 1,091 neoplastic astrocytes and in a greater fraction of neoplastic astrocytes than healthy astrocytes. This thresholding identified 5,383 cancer-specific variants, including 73 cancer-specific variants in genes found to be mutated in at least 10 TCGA GBM samples (FIG. 1C). Among these cancer-specific variants were variants in the genes EGFR and SOX9, which were previously identified as strong predictors of the neoplastic phenotype from only their expression. The mutation C3:c.4989C>T was selected based on having one of the highest neoplastic fractions of cells with variant (FCV) to perform differential expression analysis (DE) within neoplastic cells. Looking at the genes by differential expression in neoplastic cells and maximum FCV across all variants for each gene revealed EGFR, C3, COL1A2, THBS1, and LAMA5 as genes with cancer-specific variants and cancer-specific expression (FIG. 1D).

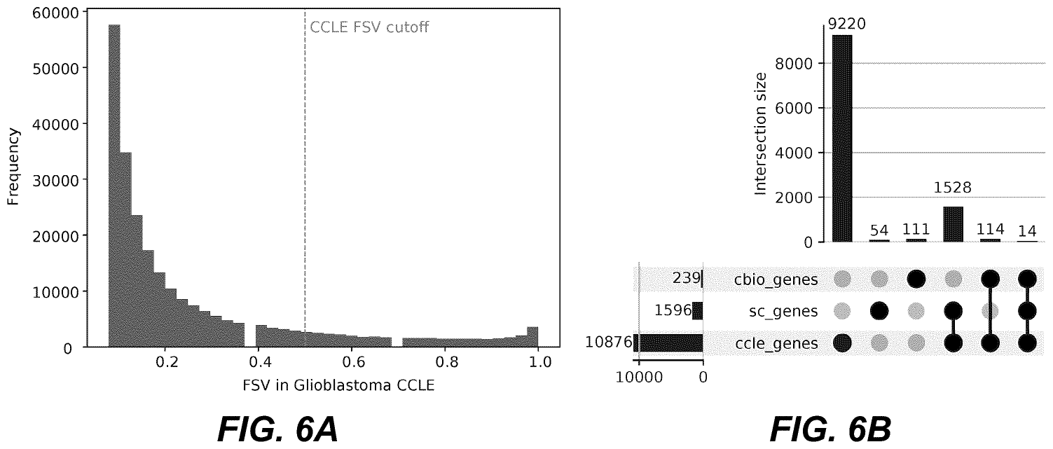

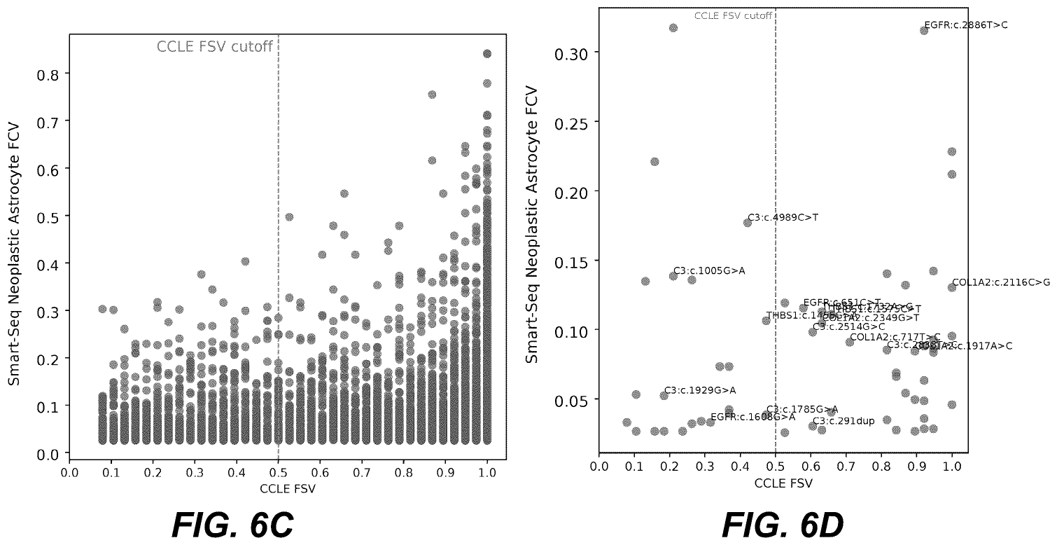

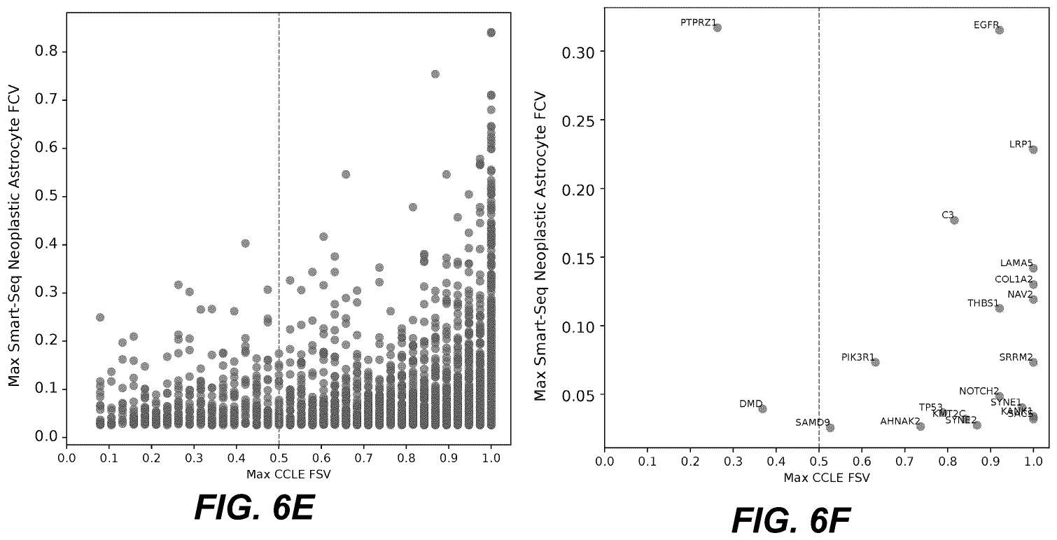

Variant calling was also performed with varseek on bulk RNA-seq GBM data from the Cancer Cell Line Encyclopedia (CCLE). Of the 5,383 cancer-specific variants identified in the GBM single-cell data, 3,719 (69.1%) were also identified in at least half of the 38 CCLE GBM samples (FIG. 1E). Some of the genes with a variant demonstrating high FCV in both scRNA-seq neoplastic cells and CCLE cells included EGFR, LRP1, C3, LAMA5, COLIA2, and THBS1 (FIG. 6A-FIG. 6F). Among the top 50 cancer-specific variants by FCV in neoplastic cells across all cell types, some variants demonstrated high frequency in myeloid and vascular cells, potentially implicating their role in angiogenesis related to tumor development (FIG. 1F). Comparing the expression of neoplastic cells with the C3:c.4989C>T mutation to neoplastic cells without the mutation revealed a strong overexpression of C3 in mutation-containing cells, followed by the IFITM3, C1R, SOD2 and CD44 genes, all known to be associated with GBM proliferation (FIG. 1G).

A varseek analysis enabled the characterization of individual cells based on the variants they possess. The total number of COSMIC CMC mutations in a cell had a positive correlation (r=0.65) with the PC1 loading value (FIG. 1H). Some of the genes that contribute the most strongly to PC1 included SPP1, C3, GFAP, and THBS1, which are involved in glioblastoma processes including hypoxia and tumor microenvironment interaction (FIG. 1I). Single-cell analysis revealed an individual cell with both high PCI and high total number of COSMIC CMC mutations that had a high number of THBS1 and SPP1 mutations, potentially indicating a more aggressive state (FIG. 1J). Some mutations tended to cluster around hotspot regions, such as the VWC and TSP 1 domains of THBS1, while others were spread more uniformly, such as in FOS (FIG. 1K).

### Varseek had High Sensitivity at Low Read Coverage

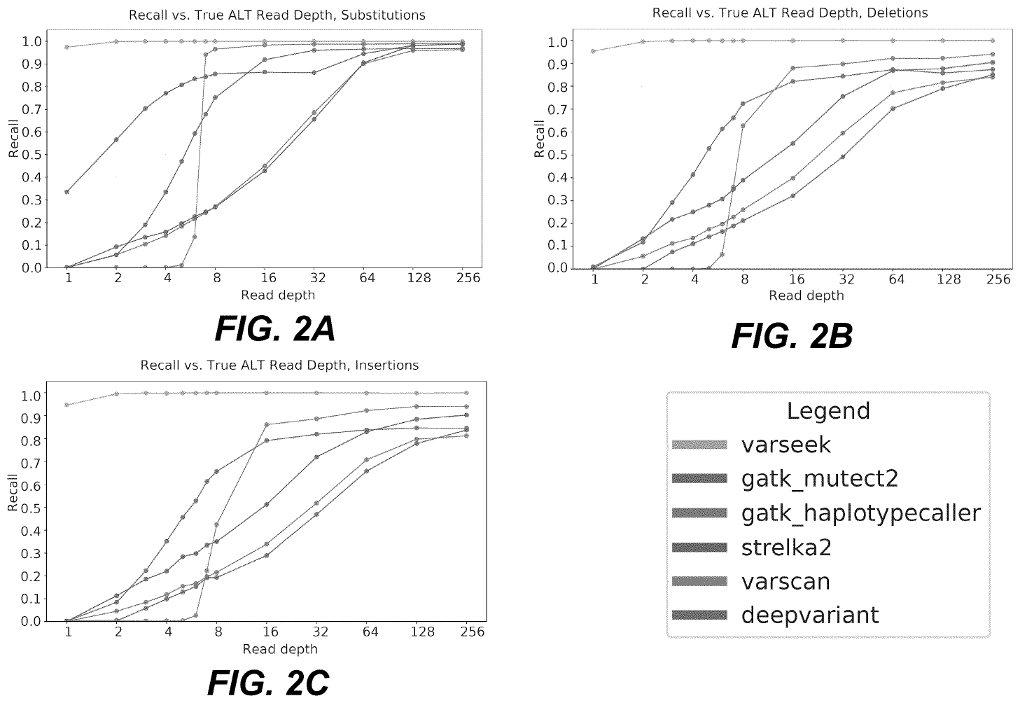

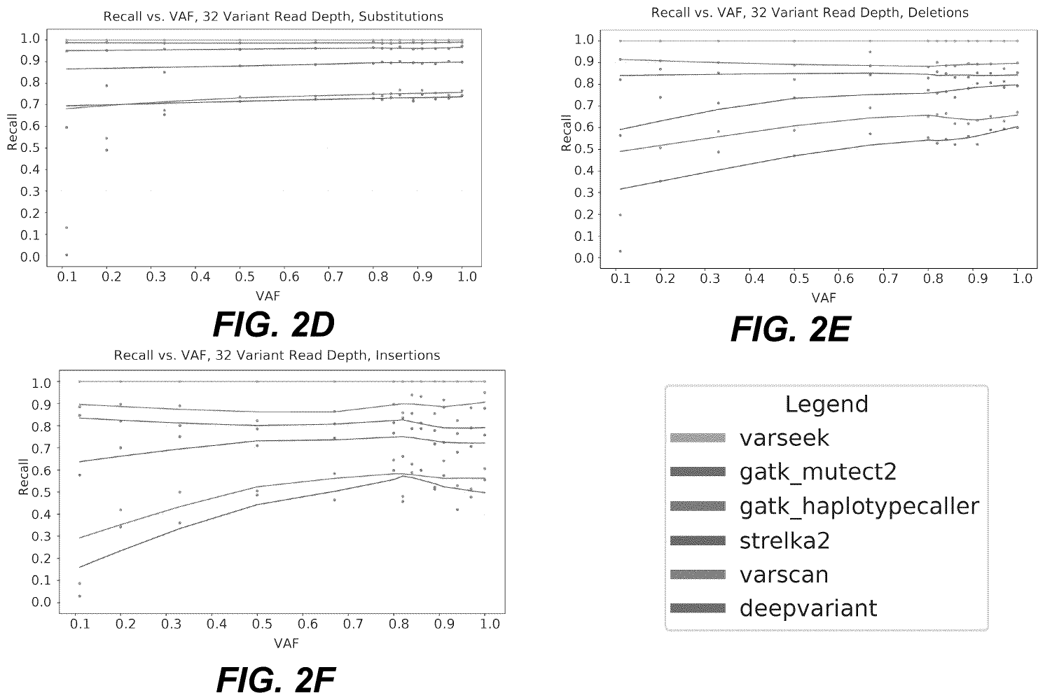

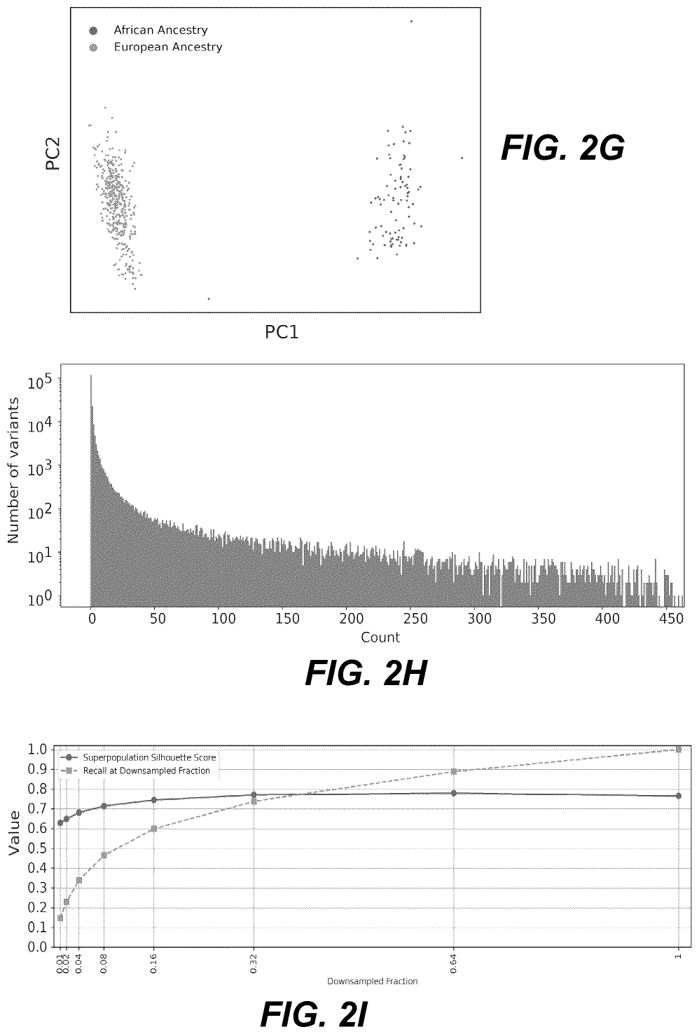

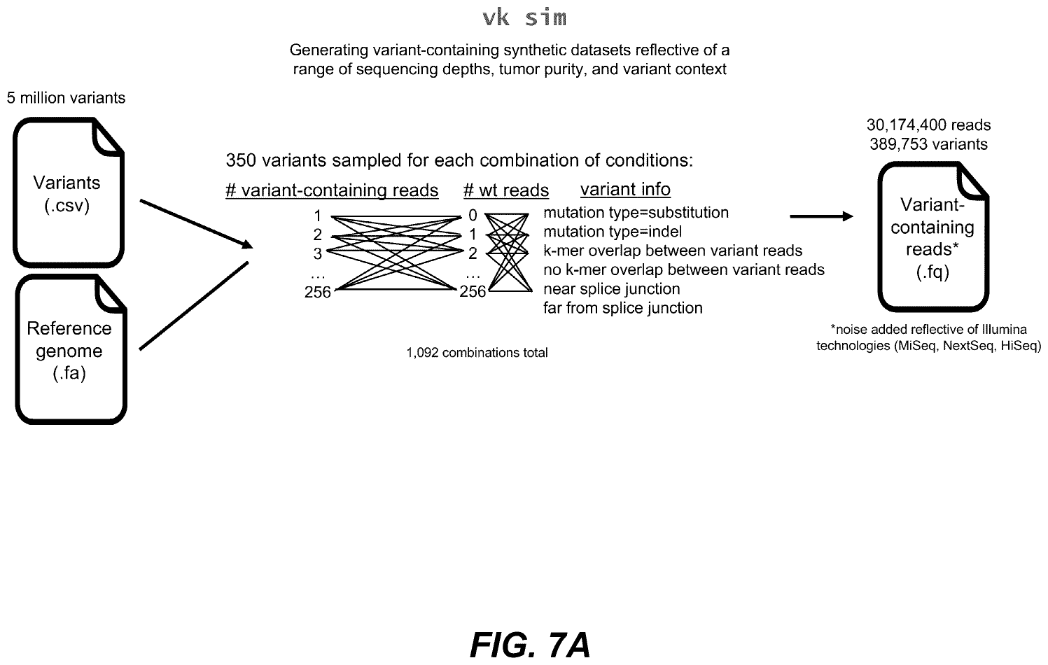

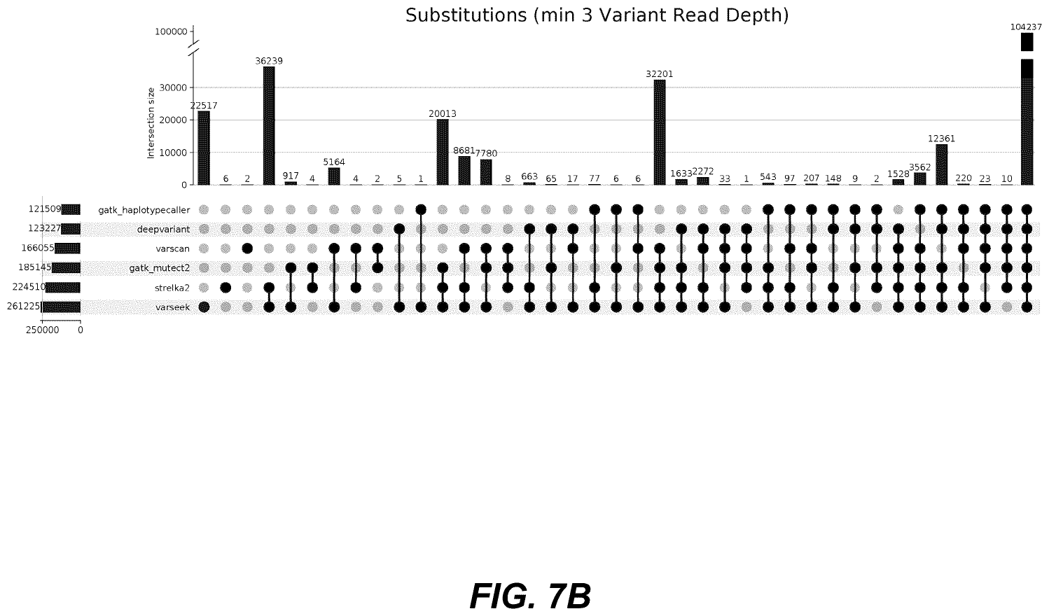

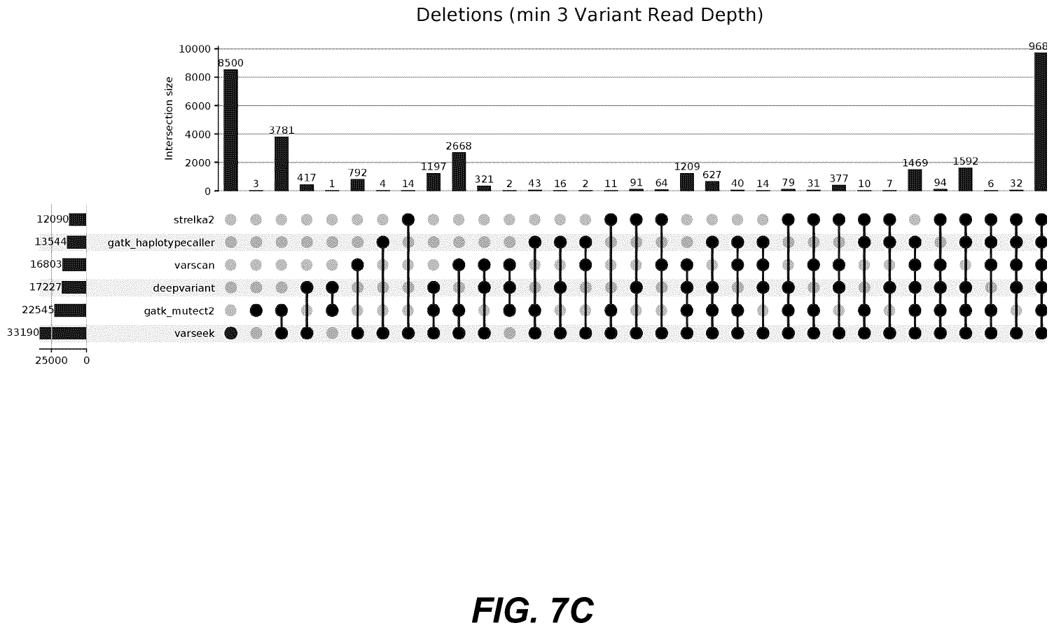

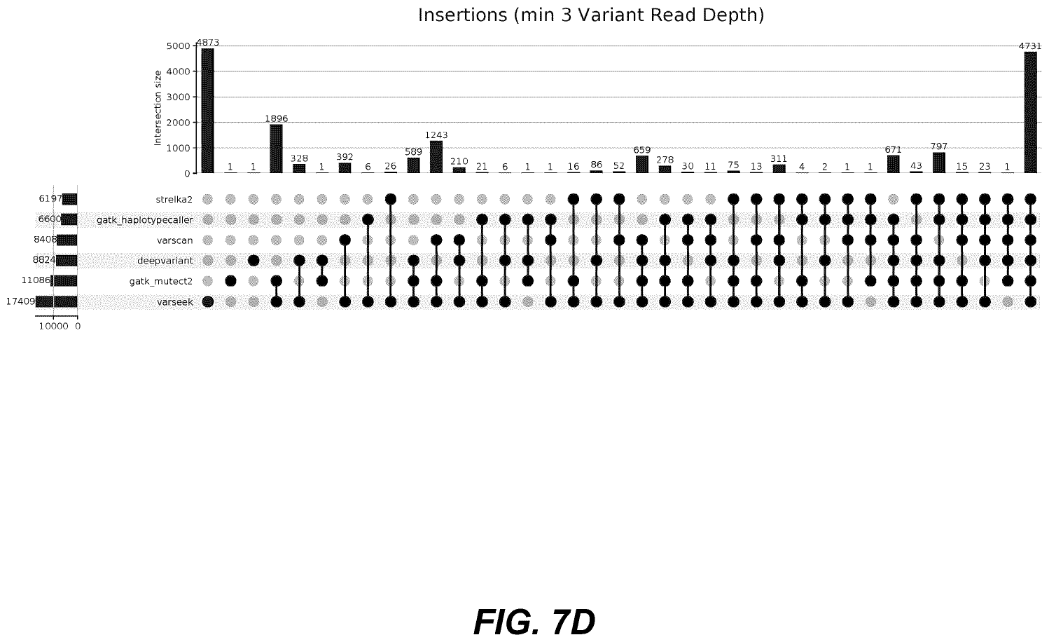

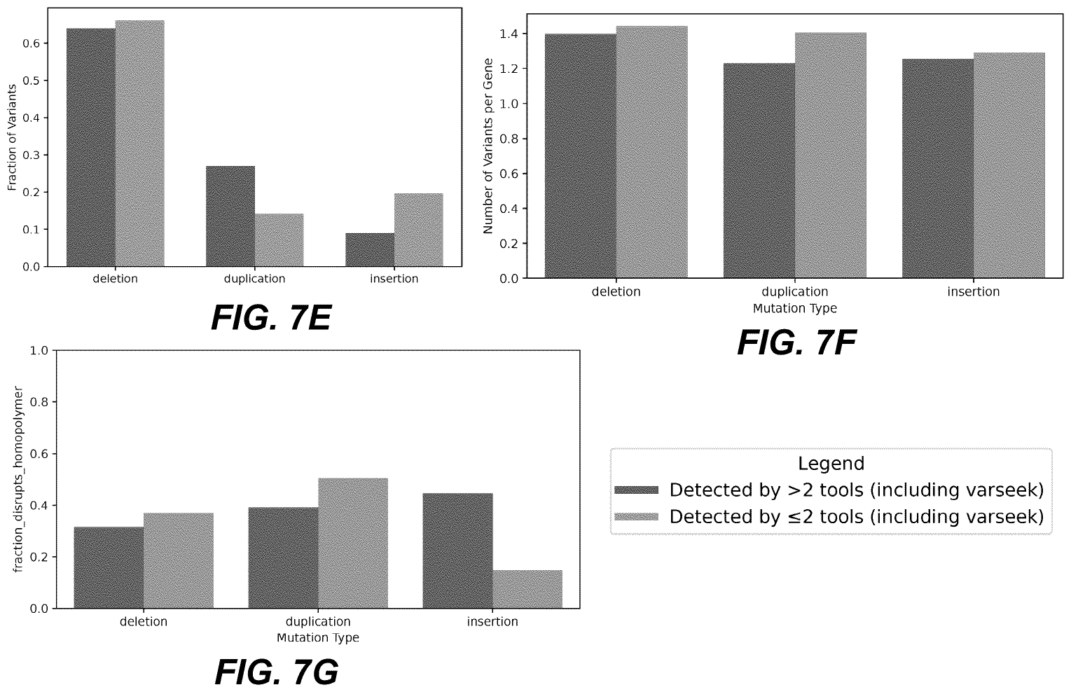

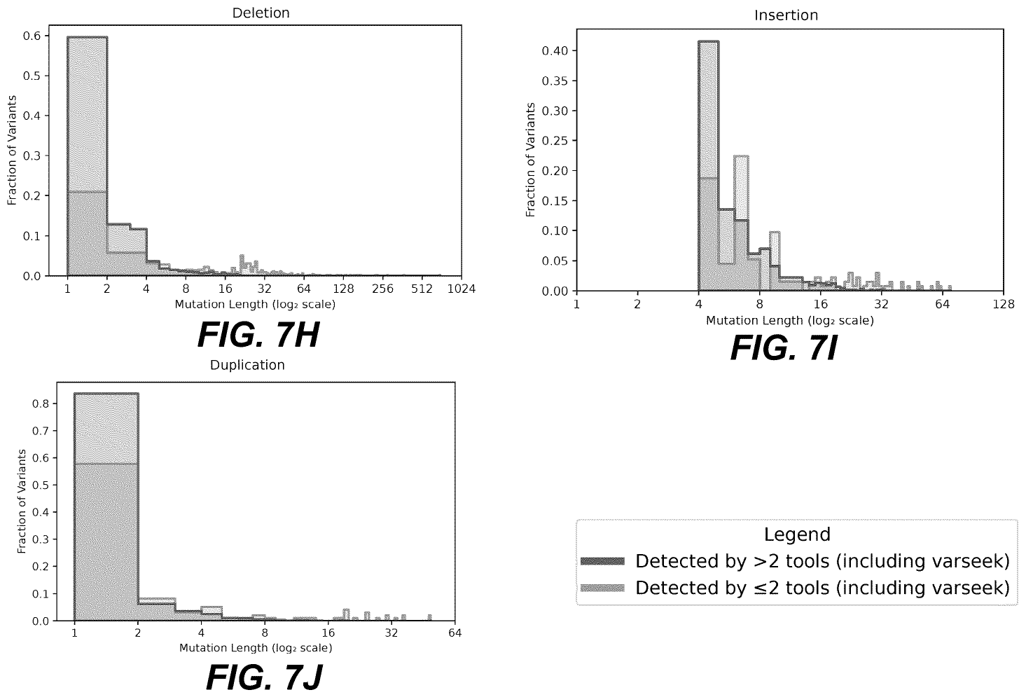

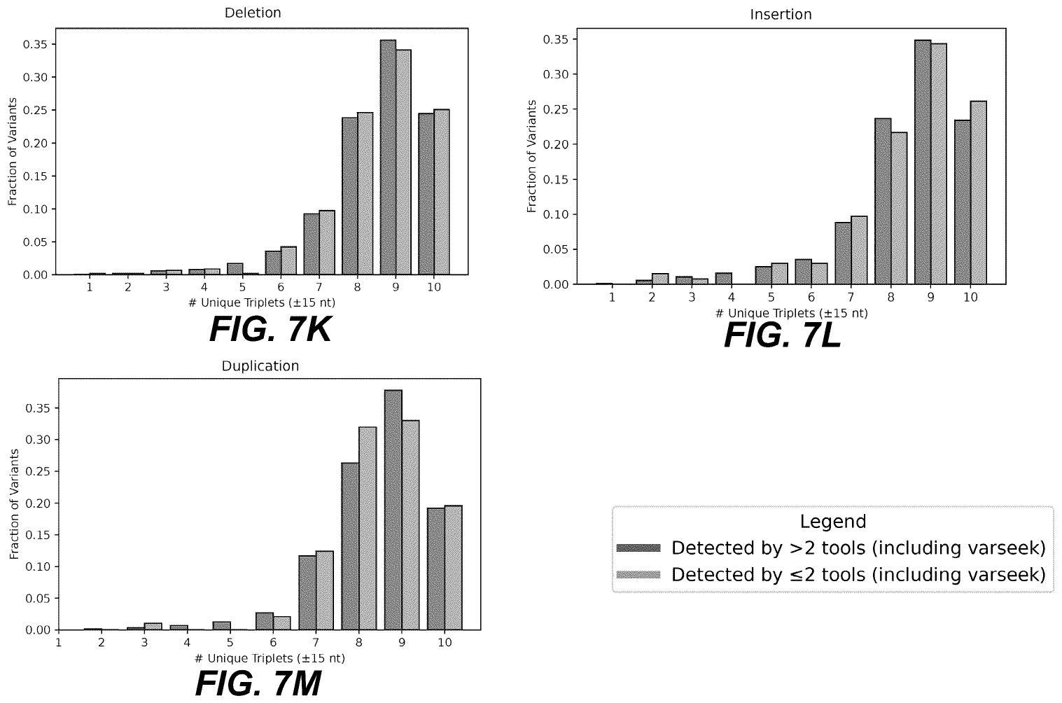

A major advance of varseek is its high sensitivity, including at low coverage. To demonstrate this, a synthetic bulk RNA-seq dataset that represented a range of variant sequencing depths, tumor purities, and variant types was developed (see Methods section) (FIG. 7A). The varseek method was compared against the most widely used variant calling tools: GATK HaplotypeCaller, Mutect2, Strelka2, VarScan and Deepvariant. Although most tools required eight or more reads to call a variant, varseek displayed high sensitivity even when there are only 2-3 variant-containing reads at a site (FIG. 2A-FIG. 2C). Furthermore, varseek retained more than 99% sensitivity across all variant sequencing depths for all variant types, while other tools required higher depths for indels than substitutions to reach high sensitivity. Variant allele frequency (VAF) had little to no effect on varseek sensitivity (FIG. 2D-FIG. 2F). All tools had precision >99% at sequencing depth ≥3 reads (data not shown).

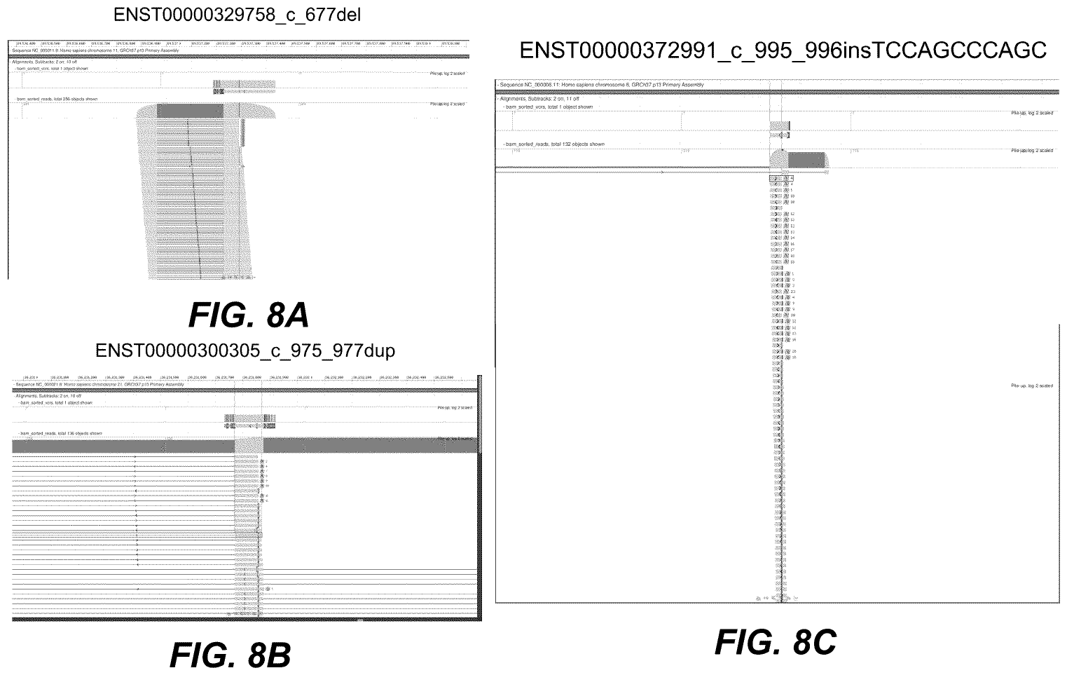

An analysis of all sites with variant sequencing depth ≥3 reads revealed that varseek only missed 35 out of 365,497 substitutions, 6 out of 33,196 deletions, and 6 out of 7,415 insertions (FIG. 7B-FIG. 7D). Moreover, varseek alone detected 22,517 out of 365,497 substitutions (6.16%), 8,500 out of 33,196 deletions (25.61%), and 4,973 out of 7,415 insertions (67.07%). These results showed that while a large percentage of variant-containing reads were correctly aligned (as they were detected by at least one tool based on read alignment), many were not detected at the variant calling step by some tools. To determine the types of indels that most other tools would not detect even at high variant sequencing depth (≥128 reads), variants detected by varseek and at most one other tool were compared to variants detected by varseek and at least two other tools. Some features that were overrepresented among poorly detected variants were insertion variants, long variants, and variant location in regions with long homopolymers (FIG. 7E-FIG. 7M). Visualization of several deletions, insertions, and duplications confirmed that even when variant calling failed in read alignment-based methods, the associated reads were generally aligned correctly (FIG. 8A-FIG. 8C).

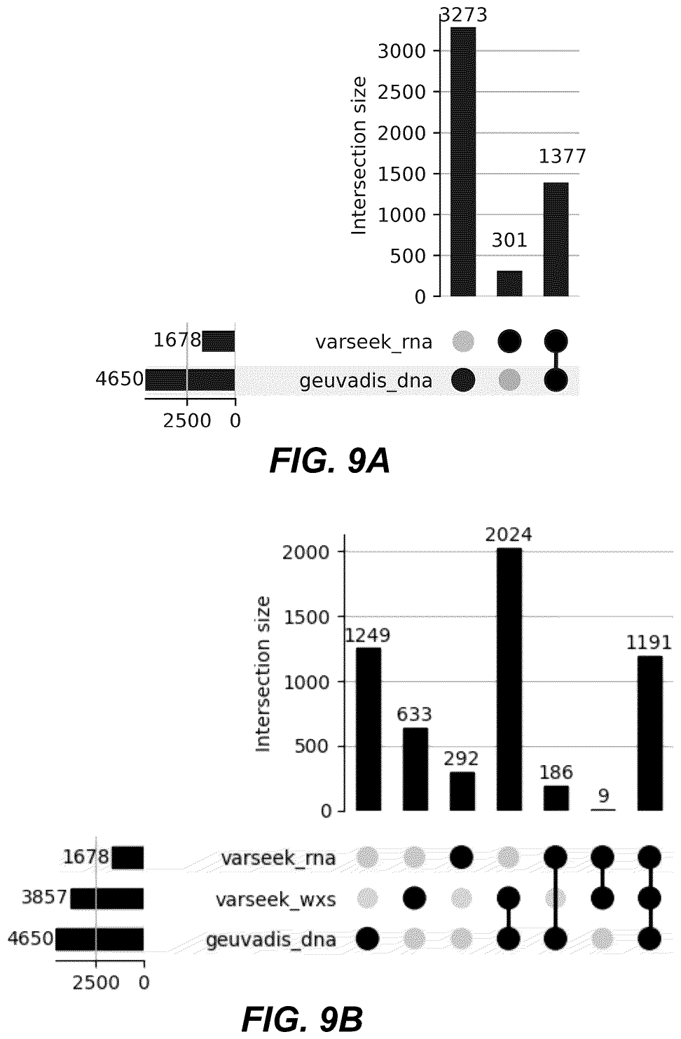

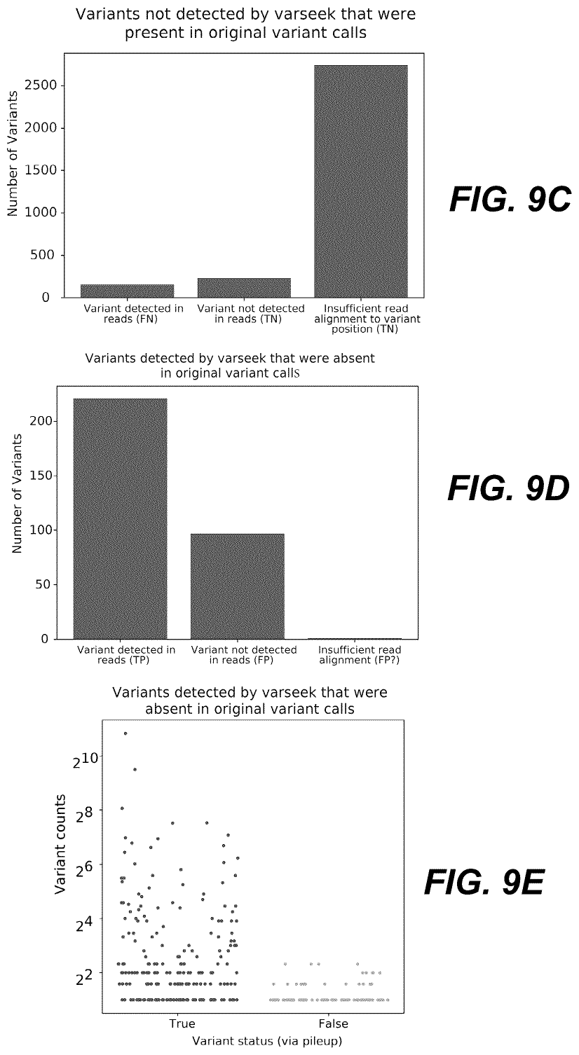

Beyond synthetic data, the sensitivity of varseek was validated on the Geuvadis whole exome sequencing (WES) and bulk RNA-seq data in FIG. 9A-FIG. 9E. A previously validated set of 203,850 dbSNP variants that were identified in the transcriptome of at least one Geuvadis sample was used as our reference variant list, a reference file was created using the vk ref command, and the Geuvadis samples were screened against this reference with vk count. As a validation, a random sample was selected and the variants detected by varseek in the RNA were compared to the variants detected by previous studies in the DNA. Most variants detected by varseek in the RNA and not detected by other tools in the DNA were either truly present in the RNA according to the read pileup (i.e., either missed by prior DNA variant calling or present as an RNA editing event) or were not truly present in the RNA and were detected in fewer than 5 reads (indicating that a count threshold of 5 would eliminate these false detections). Most variants not detected by varseek in the RNA and detected by other tools in the DNA were either not detected in the RNA according to the read pileup generated by bcftools (i.e., the non-variant allele was expressed) or did not have any reads detected (i.e., the gene was not expressed in RNA at all). vk count was run on the WES and sizable overlaps were found between varseek WES and prior DNA variant calling (likely variants not expressed in the transcriptome), as well as between varseek WES and varseek RNA (likely variants missed by prior DNA variant calling) (FIG. 9B).

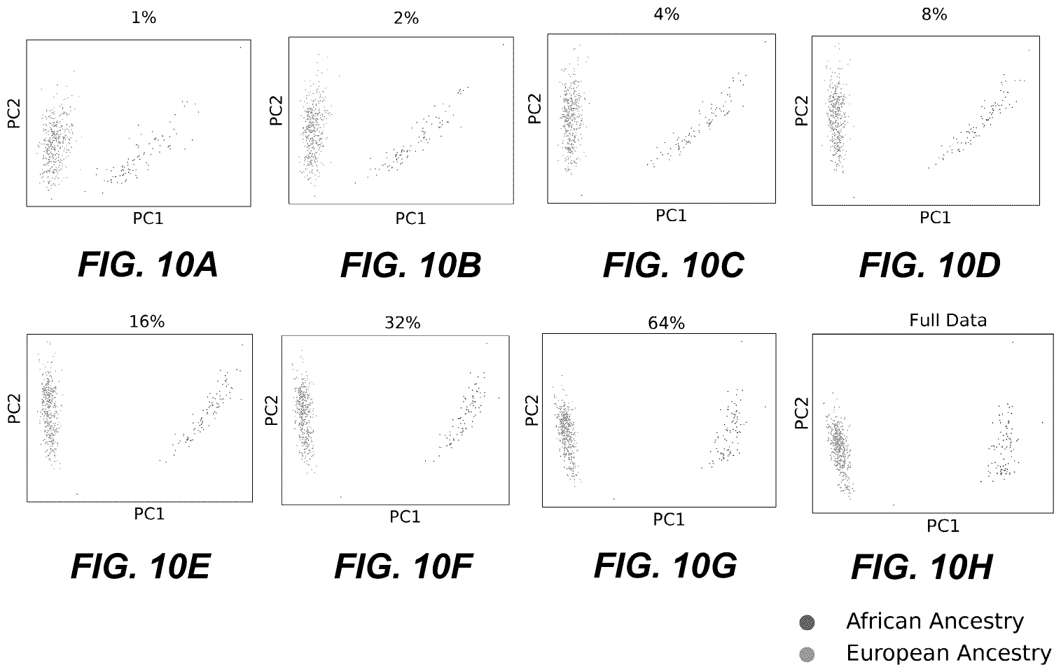

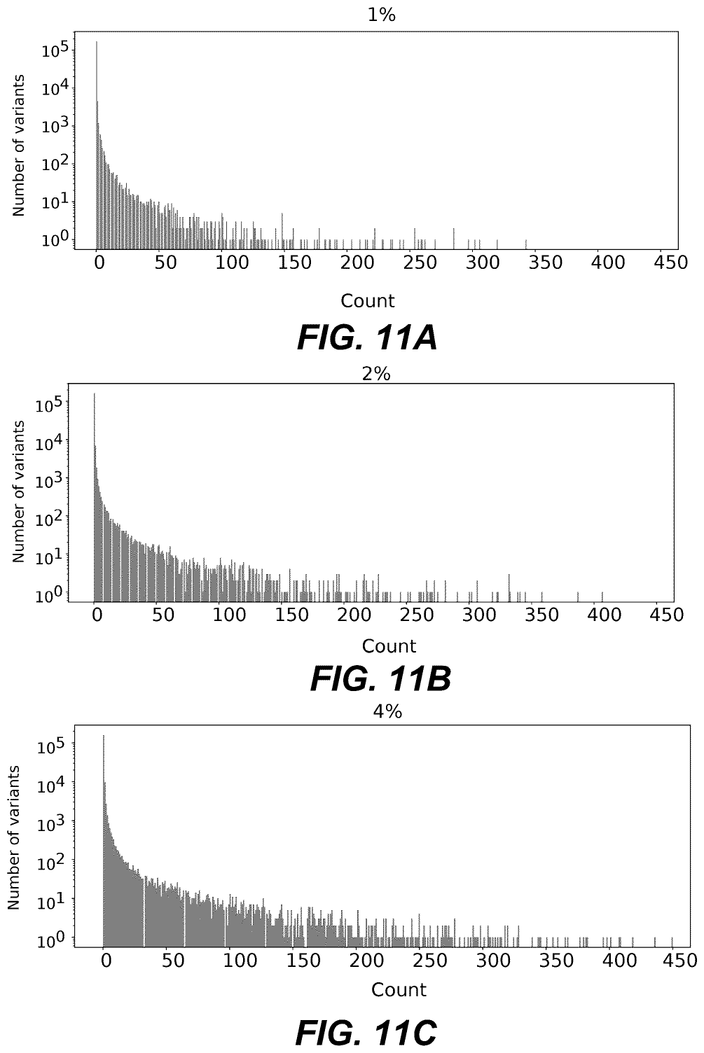

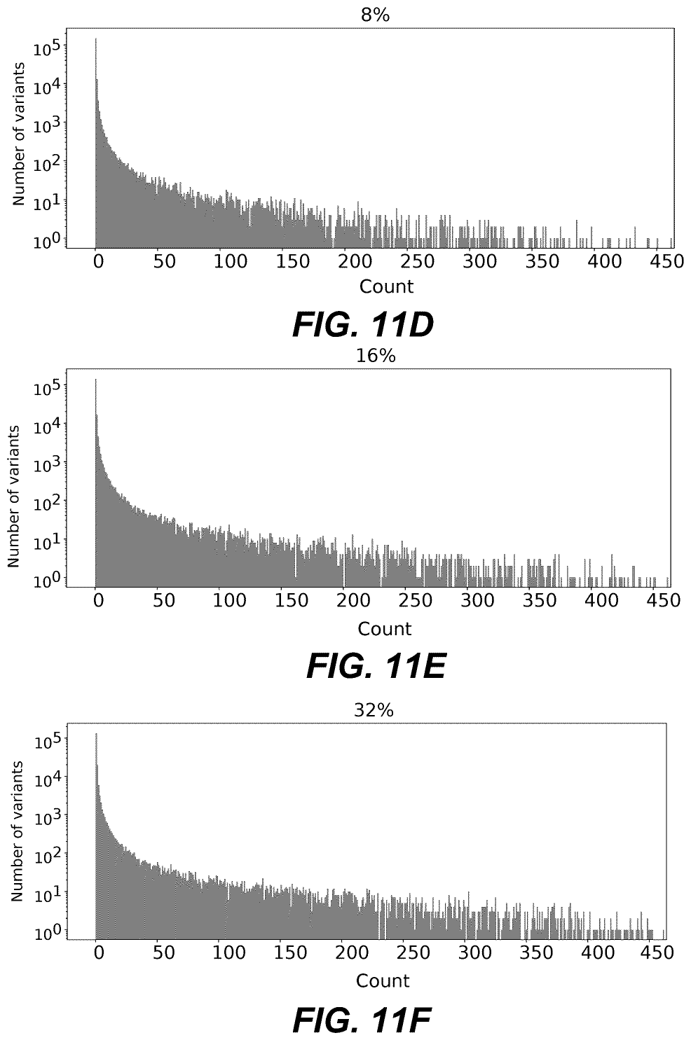

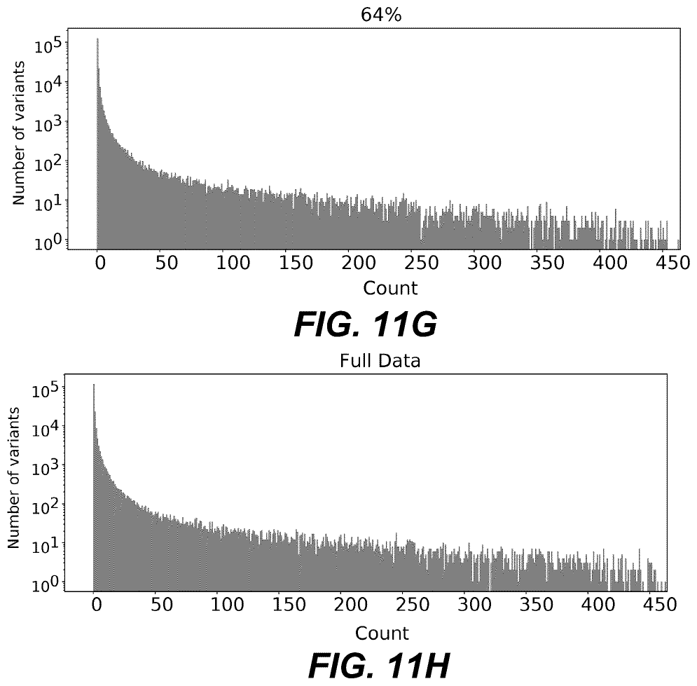

In addition to individual variant detection, the sensitivity of varseek was determined in low-coverage settings based on metrics measuring aggregate information from variants detected. The first such metric was the Silhouette Score, measuring separation of superpopulations (i.e., African ancestry vs. European ancestry) in variant PCA space. On the full dataset, these populations were cleanly separated based on their variants (FIG. 2G). The second such metric was the recall, or the fraction of true variants that were correctly detected (FIG. 2H). The sequencing read data was downsampled at various fractions, finding that the superpopulation Silhouette Score remained relatively consistent between 0.6-0.8 even at 1% of reads sampled, and the recall followed a loosely logarithmic trend (FIG. 2I, FIG. 10A-FIG. 10H, and FIG. 11A-FIG. 11H). These results demonstrated the ability of varseek to detect variants and draw meaningful conclusions in low-coverage settings.

### Varseek was Fast and Memory-Efficient

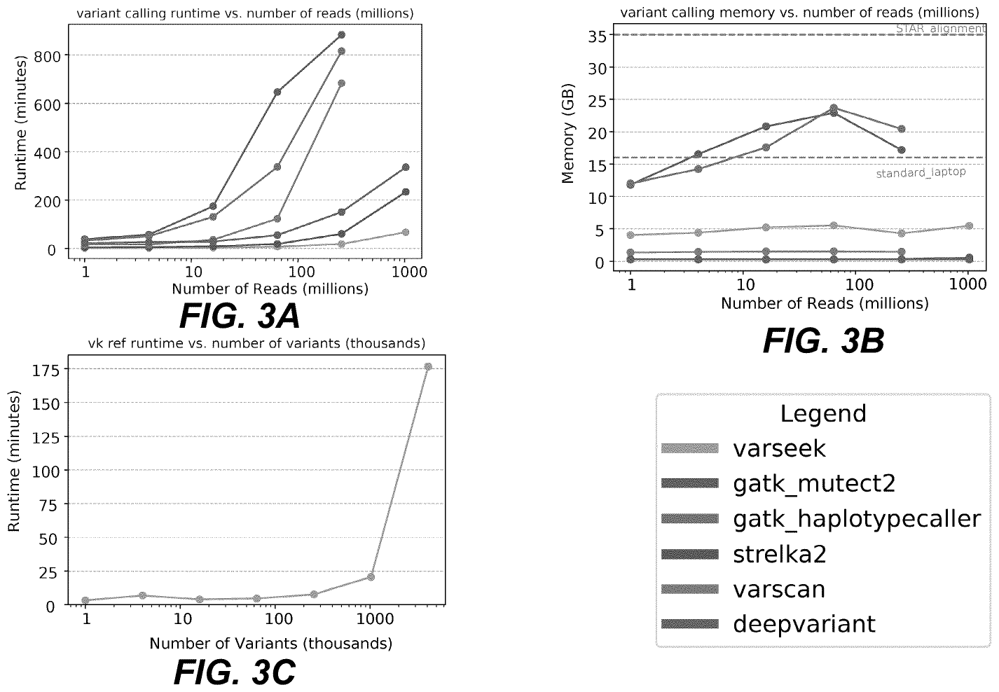

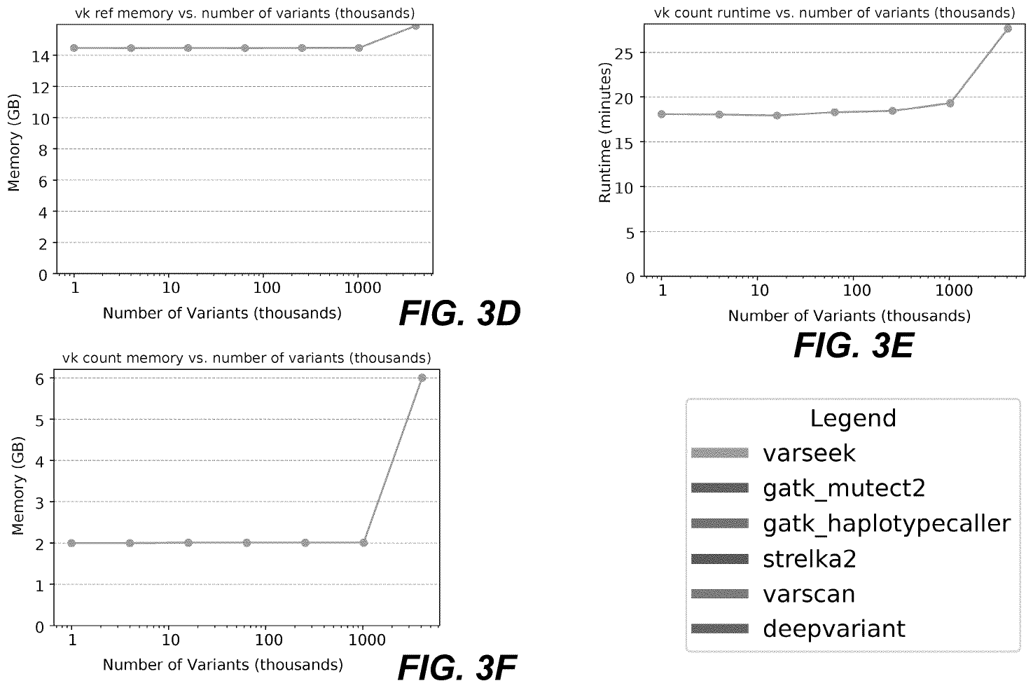

The runtime and peak memory use of varseek were compared to the same set of variant calling tools at a range of sequencing data sizes. The runtimes of all the tools increased monotonically with the number of reads. At 256 million reads, the runtimes of all tools in ascending order were as follows: varseek (18 min)<Strelka2 (58 min)<Deepvariant (149 min)<VarScan (681 min)<GATK HaplotypeCaller (804 min)<GATK Mutect2 (881 min) (FIG. 3A). Included in the times of all tools besides varseek was 31.25 minutes for read alignment to the reference genome with STAR. The peak memory usages for varseek, Strelka2, VarScan, and Deepvariant were always below 8 GB; the peak memory usages for GATK HaplotypeCaller and GATK Mutect2 were in the range of 17-25 GB for datasets of 16+ million reads (FIG. 3B). STAR alignment required a peak memory usage of 35 GB. varseek's low runtime and memory usage can be partially attributed to the optimizations achieved by kallist|bustools (kb), which included the building of a k-mer hash map for quick lookup, the creation of a colored de Bruijn graph as the index for efficient traversal, and the implementation in C. The only additional steps performed by varseek during the variant calling step involved FASTQ preprocessing with the fastp library and working with the variant count matrix with the anndata library, both of which had been largely optimized.

Additional analysis was performed on varseek to determine the relationship between variant reference size and runtime and peak memory usage. With a reference containing fewer than 1 million variants, vk ref and vk count ran at a relatively constant runtime with respect to the reference size, with vk ref running under 30 minutes and under 16 GB RAM, and vk count running under 20 minutes and under 2 GB RAM (FIG. 3C-FIG. 3F). When the number of variants in the reference surpassed approximately 4 million, runtime increased to approximately 180 minutes for vk ref and approximately 30 minutes for vk count, and memory increased to approximately 16 GB for vk ref and 6 GB for vk count. The increase in runtime of vk ref can be mainly attributed to an internal bowtie2 alignment of the variant-containing reference sequence (VCRS) k-mers to the reference genome and transcriptome. As vk ref is only run once per reference database, these runtime costs did not scale with the number of sequencing experiments to be screened.

### Discussion

The varseek method is a reference-based variant caller that enables single-cell resolution variant calling, is adaptable for use with a wide variety of assays and sequencing technologies thanks to its use of kallisto, exhibits high sensitivity, including in indel calling, and is efficient in both runtime and memory usage. Using numerous data sets, it was shown that varseek substantially simplified single-cell genetic analyses, enabling the identification of associations between variants and factors such as cell type, cancer state, and developmental stage.

k-mers can be utilized to avoid read alignment during variant calling, or to better filter. varseek benefits from utilizing a novel pseudoalignment-based approach to variant calling to overcome multiple challenges. Most of these related to false positive matches that can arise when a read shared a k-mer with a VCRS even though the read did not exhibit the corresponding variant. This situation can occur for a multitude of reasons, each of which was addressed herein:

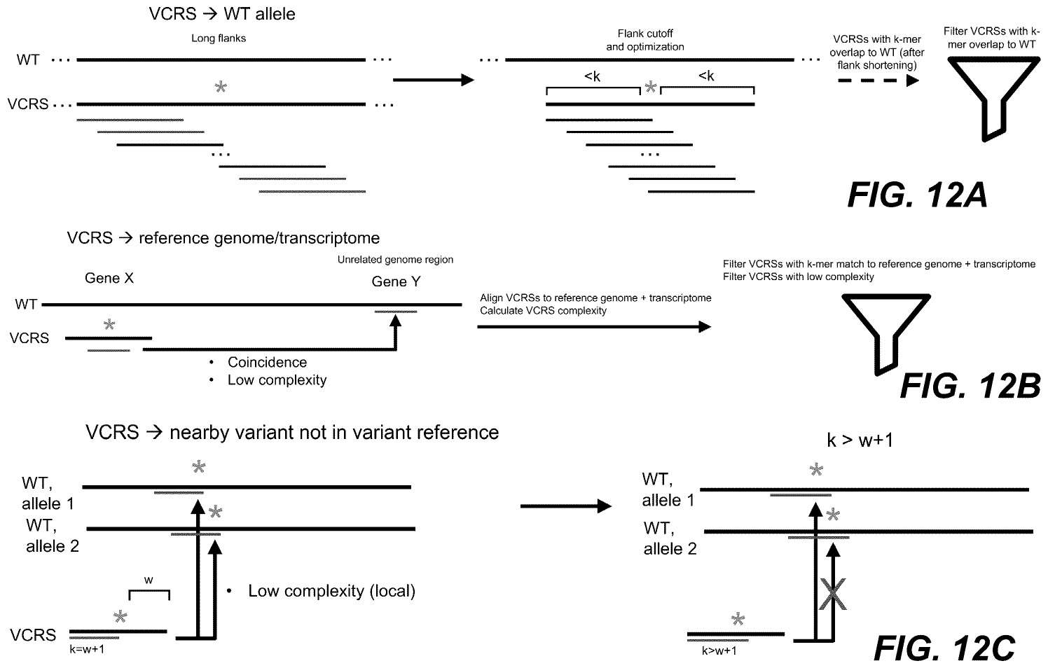

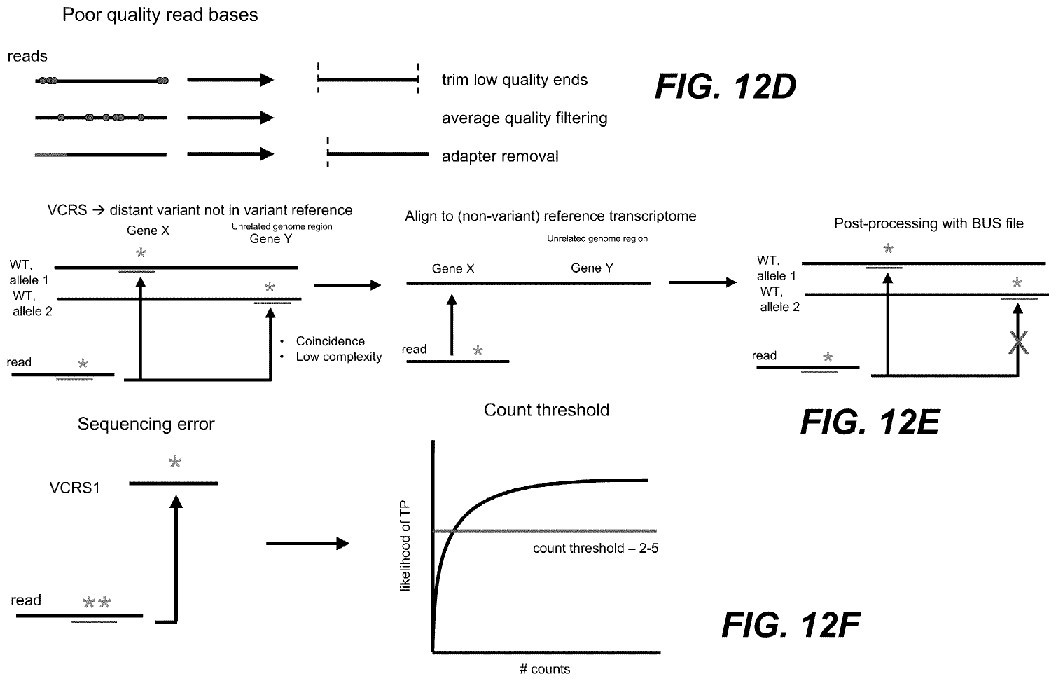

1. Sequence similarity between the VCRS and non-variant containing sequence due to flank size (FIG. 12A). Solution: Generate VCRSs such that each VCRS contains the variant region and, at most, (k−1) nucleotides flanking each side of the variant, ensuring that each k-mer contains part (if not all) of the variant.

2. Sequence similarity between the VCRS and non-variant containing sequence due to similarity between variant region and variant-flanking regions (indels only) (FIG. 12A). Solution: Implement a flank optimization procedure that shortens the beginning of the left flank based on the degree of overlap between the beginning of the variant sequence and the beginning of the right flank, and analogously that shortens the end of the right flank based on the degree of overlap between the end of the variant sequence and the end of the left flank. For VCRSs where no degree of flank shortening can remove k-mer overlap between the variant and non-variant sequence, remove these VCRSs from the variant reference.

3. Sequence similarity between the VCRS and unrelated regions of the reference genome/transcriptome (FIG. 12B). Solution: Filter out VCRSs of low complexity (long homopolymers and/or frequently-repeated nucleotide triplets), as these motifs can be repeated across the genome. Additionally, align all k-mers of all VCRSs to both the standard genome and transcriptome, and filter out VCRSs with frequent k-mer matches. As an initial conservative approach with a COSMIC reference, filter out all VCRSs with any k-mer match to any portion of the human genome or transcriptome were filtered out. In principle, some VCRSs could be retained and the runtime of vk ref could be sped up by providing a BED file for only coding regions of the genome with minimal loss of specificity.

4. Sequence overlap between the VCRS and another close-by variant (FIG. 12C). Solution: We used Use k>(w+1) to ensure that additional context beyond just the variant region is correctly identified (e.g., k=41, w=37).

5. Sequence overlap between the VCRS and a read due to sequencing errors in low-quality read regions (FIG. 12 D). Solution: Trim low-quality edges and adaptors off of reads and filter short or low-quality reads to reduce these events.

6. Sequence overlap between the VCRS and another distant variant on another gene (FIG. 12E). Solution: Align the reads to the reference transcriptome to determine the gene assignment of each read, and then adjust the variant count matrix by only keeping variant counts where the gene of the variant to which each read maps matches the gene to which each respective read maps.

7. Sequence overlap between the VCRS and a read due to occasional sequencing errors in the middle of high-quality reads (FIG. 12F). Solution: Filter out Counts from the variant count matrix below a very small minimum threshold (e.g., threshold=3).

False negatives arise when an insufficient number of variant-containing reads possess a k-mer match with the VCRS in the reference index. At the read level, these cases arise when a read contains two additional variants (whether truly occurring in the sample or from sequencing error) flanking the variant of interest on each side within k bases, or when a read contains one additional variant flanking one side of the variant of interest and there are fewer than k bases before the end of the read on the other side of the variant of interest. These false negative cases were minimized by ensuring that k was not set too large with respect to both w and the read length. Approximately 1 in every 250 bases can differ between an individual genome and the human reference genome, with a rate of closer to 1 in 1,500 bases differing in coding regions. Illumina sequencing technologies have error rates mostly lower than 1 in 1,000 bases. It was assumed that variant_frequency_rate=1/1500 uniform (rate of variation from reference genome), sequencing_error_rate=1/1000 uniform (rate of sequencing errors), k=51 (k-mer size), w=50 (VCRS window size), read_length=150 (read length), reads_present=5 (number of reads with the variant of interest), and read_threshold=3 (number of reads needed to detect to call the variant). The probability of missing a read that contained a variant was 0.006-0.033 based on its distance from the read edge. The probability of a false negative (fewer than read_threshold reads contained a k-mer match) was 0.001-0.033 due to sequencing error alone. Even if there was another variant directly preceding the variant of interest, then the variant of interest can still be detected if the variant of interest was located within the first (read_length−k) bases of the read and if the following k bases did not have sequencing errors for at least read_threshold reads. The probability of a false negative in this case was 0.271, and went down to 0.072 if the reads_present increased from 5 to 7. When building a reference of transcriptome-derived variants, the cDNA position (with untranslated regions (UTRs)) was use rather than the CDS (without UTRs) in order to provide the UTR sequences for added alignment potential. Additionally, for paired-end technologies, to ensure that distinct variants can be detected on each read of a pair, the underlying kb count command was run in single parity mode regardless of true data parity. Some of the most important parameters for balancing sensitivity and specificity included w, k, quality_control_fastqs, and qc_against_gene_matrix.

varseek can built a Variant Consensus Read Set VCRS index using a single linear reference genome, which can limit its ability to detect common variants absent in that reference. By integrating pangenomic approaches, one can construct a VCRS for each path through the pangenome graph, thus capturing variation across multiple haplotypes. For variants occurring within k bases of each other, a combinatorial indexing strategy could represent all possible combinations of closely spaced variants. When the number of such combinatorial paths becomes large, analyzing co-occurrence patterns across samples may reveal that only a subset of these variant combinations appear together, allowing reduction of the search space. Practically, after running vk ref on each path, an auxiliary column could be added to the resulting VCRS metadata dataframe that records the true variant present. This would enable post hoc collapsing of variant count matrix entries following vk count corresponding to different pangenome alignment paths that resolve to the same underlying variant, thus streamlining analysis and ensuring consistency in variant interpretation.

Furthermore, the approach disclosed herein, which addresses the challenges above, should be of independent interest as some of the strategies employed could be utilized in other read alignment and variant calling algorithms. The varseek algorithms were designed for protocols that involve short reads (10s-100s bp), such that each VCRS, which ranges in length from k to (2k−1), is comparable in length to the length of the read. This means that there is not substantially more information in a read outside of a region that may overlap with the VCRS. As long-read RNA-seq methods become more common, the k-mer-based approaches of varseek could be combined with traditional alignment-based approaches to improve the sensitivity and speed of variant calling while also utilizing the additional context in the reads.

varseek's utility has been showcased in the applications of cancer characterization. varseek can also be applied in a variety of other contexts. Any disease or genetics application for which variants of interest are useful for genotyping, diagnosis, or treatment can benefit from rapid screening with varseek. For smaller reference databases of variants, varseek can benefit from further speedups as the size of its reference index can be reduced, opening up the possibility for rapid variant calling in clinical settings. Use of current variant calling techniques that rely on all variants to be detected and subsequently screened in clinical settings can be challenging. Furthermore, varseek can be applied for variant screening in settings of low sequencing depth, including in cell-free DNA (cfDNA) or cell-free RNA screening from liquid biopsies and scRNA-seq. This is because varseek identifies variants through k-mer matching, regardless of the depth of the sequencing. varseek can also identify variants equally well in the absence of a paired healthy normal sample. Moreover, varseek can overcome the limitations of variant calling in capturing challenging variants such as gene fusions, splice variants, and RNA-editing events by characterizing the k-mers that distinguish these events from the reference genome.

There is a mature and excellent body of work on sequencing-based cancer detection that can benefit from varseek. For example, Cologuard developed a logistic regression model that combines information regarding KRAS mutations, aberrant NDRG4 and BMP3 methylation, B-actin, and a hemoglobin immunoassay from fecal cfDNA to predict colon cancer. More recent methods utilize AI approaches and multimodal data for this task. GRAIL-GALLERI is a multi-cancer screening tool that uses methylation-based analysis of cfDNA in the blood as input to a machine learning model to predict cancer, with a range of effectiveness depending on the cancer type and stage. Guardant Health's Shield test utilizes a combined genomic, epigenomic, and proteomic analysis, demonstrating 83% sensitivity and 90% specificity for detecting advanced colorectal cancer, and 13% sensitivity for advanced precancerous lesions. Gencove has shown significant promise in detecting genomic variants at very low coverage. Although methods are improving, these applications of sequencing are currently limited by the sensitivity or specificity of variant identification.

### Methods

varseek Overview

The standard varseek workflow is split into two steps: vk ref and vk count (FIG. 1A and FIG. 4). The purpose of vk ref is to create an index of variant-containing reference sequences (VCRSs) to be used as a reference for variant detection; the purpose of vk count is to screen sequencing data against this variant index. varseek utilizes the pseudoalignment algorithm from kb, implemented in kb-python, for efficient k-mer matching. Variant-containing reference sequences (VCRSs) are also referred to herein as mutation-containing reference sequences (MCRSs).

The first step, vk ref, takes as input a database of variants and the associated reference genome/transcriptome with the variant database, and produces as output an index file of VCRSs. The database of variants can be provided as a list, a variant call format (VCF) file, or a CSV/TSV file with a column for sequence ID and a column for variant information in a format complying with Human Genome Variation Society (HGVS) guidelines. The reference genome/transcriptome can be provided as a list or FASTA file. Each VCRS is designed such that each k-mer (subsequence of length k) of each VCRS contains at least one variant nucleotide while also maximizing sequence length. Trimming and filtering of VCRSs are applied to minimize false positives, and recommended argument values are provided to balance between sensitivity and specificity (more details in the Discussion section). The variant index file is generated from a FASTA file containing the VCRSs via kb ref. This index-generation step need only be performed once for a variant database, as the index file can be reused across sequencing experiments.

The second step, vk count, takes as input the index of VCRSs produced by vk ref and sequencing data, and produces as output a cell/sample x variant matrix. The sequencing data can be provided as a FASTQ file. Trimming and filtering of the sequencing read data are applied, and validation and thresholding of the cell/sample x variant matrix are applied to minimize false positives. As with vk ref, recommended argument values are provided to balance between sensitivity and specificity (more details in the Discussion section).

### Kallisto Pseudoalignment

The core of the variant screening methodology disclosed herein includes of the k-mer-based pseudoalignment approach underlying kallisto. To summarize this pseudoalignment approach, kallisto first builds an index from the reference transcriptome including a hash map that maps all k-mers from the reference transcriptome to the position of the node corresponding to that k-mer in a de Bruijn graph for the reference. The kallisto suite of tools also creates a t2g file that maps transcript names to their corresponding gene names. During alignment, kallisto breaks down sequencing reads into k-mers of the same size k, identifies the matching k-mers from the De Bruijn graph, and creates an equivalence class (a set of all viable potential transcript mappings) based on taking the intersection of perfect k-mer matches across all k-mers in the read. The set of transcripts in the equivalence class is then mapped to the unique set of corresponding genes. All ambiguous bases (Ns) in the reads are replaced with As; all ambiguous bases in the reference are replaced with a random base drawn from the uniform distribution. If an equivalence class corresponds to multiple genes, then this read is considered to be multimapped. If the intersection of k-mer matches in a read is the empty set (either if there is not a single k-mer match between a read and the reference de Bruijn graph, or if different sets of k-mers map to different transcripts), then that read is considered to be unmapped. For varseek, in some embodiments by default, the variants that correspond to the union, rather than the intersection, of k-mer matches, are taken as this allows variant detection to be independent of which variants comprise the reference file. This was balanced by increasing the default k, from 31 for standard kallisto to 51 for standard varseek in some embodiments, as well as providing the option to validate that a read needs to be pseudoaligned to the same gene as the gene of the variant to which it maps.

### Variant Reference Creation and Filtering

To build the variant reference, vk ref, which wraps around vk build, vk info, vk filter, and kb refis (FIG. 4), can be called. FIG. 5 shows a flowchart that details the variants retained starting from the COSMIC CMC database and ending with the final variant reference used in analysis.

vk build takes as input the variant database (as a CSV/TSV file, a VCF file, or a list) and the reference genome from which the variants were annotated. For each variant in the database, it refers to that region of the reference genome, induces the variant, and adds the variant region and w nucleotides flanking each side of the variant region to the VCRS FASTA output. The argument value for w needs to be strictly less than the argument value for k, as this ensures that each k-mer in each VCRS contains the variant. As w decreases in magnitude relative to k, there becomes a greater required overlap on each end of the VCRS to match k-mers during alignment. In some embodiments, default values are set for w=47 and k=51. To ensure that VCRSs do not share k-mers with their wild type (WT) counterparts, a sequence flank shortening algorithm is implemented, wherein the left flank is shortened by the number of nucleotides overlapping between the beginning of the variant region and the beginning of the right flank, and analogously, the right flank is shortened by the number of nucleotides overlapping between the end of the variant region and the end of the left flank. VCRSs that shared a k-mer with their WT counterpart despite flank shortening would be discarded, which accounted for <0.001% of sequences in the COSMIC CMC reference and were generally of low sequence complexity. The headers of VCRSs would be merged when they were identical to each other or one another's reverse complement (relevant when the sequencing data is unstranded), as these sequences are indistinguishable in terms of k-mer matching. The entire vk build module is entirely vectorized with the pandas Python library.

vk info computes statistics regarding the variant reference and stores these in a dataframe, which can be input to vk filter to filter the variant reference as desired. The statistics that vk info can compute include the following:
nucleotide composition
sequence length (mean, total, minimum, maximum)
whether or not the spliced and unspliced VCRS are identical
distance to the closest splice junction
number of unique transcripts and number of unique genes
the set of nearby variants for each variant
the number of k-mer overlaps to the normal reference transcriptome and genome
the number of k-mer overlaps to other VCRSs
longest homopolymer length
triplet nucleotide complexity (the number of distinct triplets divided by the number of total triplets)
whether or not the VCRS is a superstring or a substring of another VCRS.

In some embodiments, by default, the only computed statistics are (1) the VCRS k-mer alignment and pseudoalignment to the reference genome and transcriptome, (2) longest homopolymer length, and (3) triplet nucleotide complexity.

vk filter filters VCRSs from the FASTA file based on a list of provided filters in the format COLUMN: RULE or COLUMN: RULE-VALUE, where COLUMN is a field in the metadata table, RULE is a rule (valid choices follow), and VALUE is the VALUE to match. Supported rules included numeric comparisons (greater_than, greater_or_equal, less_than, less_or_equal, between_inclusive, between_exclusive), percentile thresholds (top_percent, bottom_percent), equality tests (equal, not_equal, is_in, is_not_in), Boolean checks (is_true, is_false, is_not_true, is_not_false), and nullity (is_null, is_not_null). Only the VCRSs passing all filters remain in the filtered FASTA file. In some embodiments, by default in the vk ref pipeline, the applied filters are that only the VCRSs without a k-mer alignment to the reference genome or transcriptome, with five or fewer distinct nucleotide triplets, and without a homopolymer of greater than 10 bases in length.

The filtered VCRS FASTA file is passed into kb ref with a custom workflow to generate the VCRS index used for pseudoalignment.

### Variant Calling

To screen sequencing data against the variant reference, varseek uses the varseek count command, which wraps around vk fastqpp, kb count, varseek clean, and varseek summarize (FIG. 4)

vk fastqpp performs preprocessing on the FASTQ files. In some embodiments by default, all processing is turned off. However, if FASTQ process have not been quality controlled, it is recommended to set ‘quality_control_fastqs,’ ‘cut_front,’ and ‘cut_tail’ all to True. ‘quality_control_fastqs’ enables quality control by applying the fastp package to trim low quality edges and adapters, filter sequences by average base quality and length, and perform additional read processing. While fastp is designed for DNA and bulk RNA data, the package has been extended to work for barcoded single-cell data by ensuring that read processing only occurs on the file(s) and region(s) of each file with cDNA sequence (as opposed to barcode, UMI, etc.), and that any additional files with barcode/UMI information are adjusted accordingly to ensure synchronization. vk fastqpp also includes the argument ‘split_reads_by_Ns_and_low_quality_bases,’ which splits reads by Ns and low-quality bases to minimize false alignment due to poor sequencing. This argument is generally not recommended, as it can introduce a high rate of false negatives due to the shearing of reads.

kb count pseudoaligns the FASTQ read data to the reference variant index. In some embodiments by default, the multimap and union flags are used, as one read can contain multiple variants, and the detection of any variant in the index should be independent of the detection of any other variant. To ensure that each read in paired-end data can have distinct variants detected, kb count is run in single-stranded mode for paired-end data and then the resulting matrix file is corrected to collapse barcodes across pairs. An additional kb count command can pseudoalign the FASTQ read data to the reference genome index if downstream steps require this.

vk clean takes as input the Anndata matrix file(s) generated from the kb count step and processes these data. Two of the most important parameters of vk clean are ‘qc_against_gene_matrix’ and ‘min_counts.’ ‘qc_against_gene_matrix’ cross-references variant alignment by looping through each read, checking its alignment to the variant reference and gene reference, and ensuring that the gene of the variant to which the read maps in the variant reference matches the gene to which the read maps in the gene reference. This step is off by default in some embodiments. ‘min_counts’ performs thresholding on the count matrix to eliminate variant calls at low counts that have a high likelihood of being attributed to sequencing error. vk clean also includes a series of parameters to perform standard scRNA-seq processing on the variant matrix based on information from the gene matrix, including cell and gene count thresholding, doublet detection, and normalization, all of which are off by default.in some embodiments

‘vk summarize’ produces a summary text file with some basic analysis on the variant count matrix. Some of these results include the number of variants detected in each cell/sample, the variants detected across the most cells/samples, and the genes with the most variants detected. This step is off by default in the vk count command in some embodiments.

Comparison of varseek to Other Variant Calling Tools

All benchmarked variant calling tools required the generation of a BAM file as the first step in the variant calling pipeline. This was performed using STAR with splice-aware settings, aligning to GRCh 37, Ensembl release 93 to agree with the COSMIC reference.

The GATK variant calling pipeline followed was the “RNAseq short variant discovery (SNPs+Indels)” best practices guideline. To briefly summarize this process, after performing alignment with STAR, the FASTQ data was converted to an unmapped BAM for input to MergeBamAlignment; MarkDuplicates was run to identify duplicate reads; SplitNCigarReads was run to split reads by alignment gaps over introns; BaseRecalibrator was run to recalibrate the base qualities using information from the alignment and known sites from the 1000 genomes common SNP VCF; and then HaplotypeCaller and Mutect2 were run on the processed BAM file to perform variant calling. All filtering was disabled for each of these tools in order to prevent incorrect filtering that would arise from the synthetic nature of the dataset.

For Strelka2, variant calling was configured in RNA mode (−ma) with default germline workflow parameters. The workflow was set up via configureStrelkaGermlineWorkflow.py and executed locally with multi-threading enabled. For VarScan, pileup files were generated using samtools mpileup with base alignment quality recalculation disabled (−B) and no base quality filtering. Variants were called with VarScan mpileup2cns using minimal filtering thresholds (minimum coverage of 1, at least 1 supporting read, variant frequency ≥1%, and no strand bias filter), outputting VCF format. For Deep Variant, variant calling was performed in accordance with a RNA-seq case study. Mosdepth was used to compute per-base coverage of aligned reads, followed by filtering positions exceeding the minimum coverage threshold (set to 3) with bedtools. Variant calling was performed using a downloadable model trained on RNA-seq data, enabling read splitting (split_skip_reads=true) and running with full CPU parallelism to generate VCF outputs.

In order to map the detected variants to the COSMIC database, the COSMIC CMC mutations were converted to a VCF format. Both the COSMIC ground truth VCF and output VCF of each tool were normalized with ‘bcftools norm,’ and VCFs were compared with hap.py.

### Synthetic Data Creation

Synthetic bulk RNA-seq data was created with a series of calls to vk sim. A reference of read parents was first built with vk build that contained each variant and the 149 nucleotides flanking each side of the variant. Reads were selected for each combination of conditions from selections 1-3 below:
Variant sequencing depth of m=[1, 2, 3, 4, 5, 6, 7, 8, 16, 32, 64, 128, 256]
WT sequencing depth of w=[0, 1, 2, 3, 4, 5, 6, 7, 8, 16, 32, 64, 128, 256]
Variant type of substitution, variant type of non-substitution, contains k-mer overlap in the VCRS reference, does not contain k-mer overlap in the VCRS reference, is within 10 bases of a splice junction, is farther than 10 bases from a splice junction.

For each combination of conditions, 350 variant read parents were randomly selected from the COSMIC CMC mutation reference that were not already included in the synthetic data. Then, m different starting positions were selected between (read_parent_length−read_length+1), overlapping some starting positions only if m was greater than this value, and reads of length 150 starting at each point were created. A similar procedure was repeated for “wildtype” (WT) reads (i.e., non-variant containing reads), drawing from a reference of WT read parents (sequence fragments which had the same start and end points as the mutant fragments but without the variant introduced). Random variants-85% substitutions, 10% single nucleotide deletions, 5% single nucleotide insertions-were introduced in the reads at a rate of 1 in 1000 bases, according to standard Illumina error rates. 10,000 reads drawn from random regions of the transcriptome not overlapping with any of the COSMIC CMC variants were also added. All base qualities were set to the highest Phred33 score of I.

### Glioblastoma Data Analysis

A reference variant was built from the COSMIC CMC cDNA with vk ref using default arguments. Variant calling was performed with vk count using standard arguments on Smartseq2 data previously published and bulk RNA-seq data from the CCLE.

### Geuvadis Data Analysis

A reference variant index was built from a set of variants previously identified by GATK HaplotypeCaller. These variants were filtered only to retain those in exon regions. dbSNP IDs were used to convert from HGVSg to HGVSc notation, and the GTF file was used to convert from coding DNA sequences (CDS) to cDNA positions. As the original variant calls were generated by plink, the REF and ALT alleles were validated based on the dbSNP information. vk ref was run on these variants with w=37, k=41, and otherwise default arguments. Variant calling was performed with vk count using standard arguments (except k=41) on the Geuvadis bulk RNA seq data and whole exome sequencing data.

### False Negative Calculations

False negative (FN) risk was quantified under realistic sequencing noise and background variation. For each read, a flank of length k−1 adjacent to the mutation of interest was modeled and two independent per-base hazards were treated: (i) a natural background variant with rate v and (ii) a sequencing error with rate s. For a conservative per-base hazard, p≈v+s (rare-event union bound) was used. Thus, the probability that a flank was clean was (1−p)^{k-1}. An upper bound on FN was reported by requiring a clean flank on only one side (the case when the variant of interest was on the end of the read), and a lower bound by requiring clean flanks on both sides (the case when the variant of interest was in the middle of the read), i.e., (1−p)^{k-1} vs. (1−p)^{2(k-1)} (with analogous decompositions using v alone or e alone to isolate biological variation vs. sequencing error). In the no linked-variant scenario, read count variability was modeled by drawing the number of sequenced reads from a binomial distribution; under a worst case, it was assumed that all informative reads required a single clean flank (one-sided), and under a best case, they required two clean flanks (two-sided). For each draw, the probability of achieving at least a user-specified threshold of “good” reads was computed and FN was summarized as the complement. In the linked-variant scenario (a second variant immediately upstream), a read was usable only if (a) the focal variant landed in a valid position—probability (read_length−k)/read_length—and (b) the following k−1 bases were error-free-probability (1−s)^{k-1}. The per-read variant detection probability was thus

$${P}_{det} = \frac{read\_length - k}{read\_length} {( 1 - s )}^{k - 1} ,$$

and, given coverage n, the probability of observing at least the read threshold was the binomial tail Pr[X≥t], X˜Binom (n, P_{det}), with FN equal to 1−Pr[X≥t]. Data Visualization

Pileup visualizations were performed with the NCBI workbench. Lollipop plots were created with the lollipops package. All other visualizations were made with matplotlib.

### Determining One or More Variants

Disclosed herein include embodiments of a method for determining one or more variants (e.g., mutations). In some embodiments, a method for determining one or more variants (or one or more steps thereof) is under control of a processor (e.g., a hardware processor or a virtual processor). The method can comprise: receiving variant information of a plurality of variants and an associated reference. The method can comprise: generating a plurality of variant-containing reference sequences (VCRSs) from the variant information of the plurality of variants and an associated reference. The method can comprise: receiving a plurality of sequence reads obtained from a sample. The method can comprise: determining one or more variants present in the plurality of sequence reads using the plurality of VCRSs. In some embodiments, the method can comprise: building an index of the plurality of VCRSs. Determining the one or more variants present in the plurality of sequence reads can comprise: determining the one or more variants present in the plurality of sequence reads using the index of the plurality of VCRSs

In some embodiments, receiving the variant information of a plurality of variants comprises: receiving the variant information of the plurality of variants from a database of variants, optionally wherein the database of variants comprises Catalog Of Somatic Mutations In Cancer (COSMIC). In some embodiments, the reference associated with the variant information of the plurality of variants is the reference from which the plurality of variants are annotated. In some embodiments, the reference comprises a reference genome or a reference transcriptome.

In some embodiments, a VCRS (or one or more VCRS or each VCRS) of the plurality of VCRSs comprises a variant region flanked by a plurality of nucleotides (w) flanking each side of the variant region. w can be an integer. w can be different in different implementations. For example, w can be, be about, be at least, be at least about, be at most, or be at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number of a range between any two of these values. In some embodiments, w is strictly less than k. In some embodiments, a VCRS (or one or more VCRS or each VCRS) of the plurality of VCRSs comprises a variant region flanked by a first plurality of nucleotides (w1) flanking left side of the variant region and a second plurality of nucleotides (w2) flanking right side of the variant region. w1 can be an integer. w2 can be an integer. w1 and/or w2 can be different in different implementations. For example, w1 and/or w2 can be, be about, be at least, be at least about, be at most, or be at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number of a range between any two of these values. w1 can be bigger than w2. w1 can be smaller than w2. w1 and w2 can be identical. In some embodiments, w1 is strictly less than k. w2 is strictly less than k.

In some embodiments, the method comprises: shortening a sequence flank (such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more, nucleotides) of one or more VCRSs of the plurality of VCRSs. In some embodiments, the method comprises: trimming one or more VCRSs of the plurality of VCRSs prior to building the index of the plurality of VCRSs. In some embodiments, the method comprises: comprising filtering one or more VCRSs of the plurality of VCRSs prior to building the index of the plurality of VCRSs.

In some embodiments, each VCRS (or a VCRS or one or more VCRSs) corresponds to a variant (e.g., a different variant) of the plurality of variants. In some embodiments, each k-mer (or a k-mer or one or more k-mers) of a VCRS of the plurality of plurality of VCRS comprises at least one variant nucleotide (such as 1, 2, 3, 4, 5, 6, 7, 8, 9, 10, 11, 12, 13, 14, 15, 16, 17, 18, 19, 20, or more) of the corresponding variant of the plurality of variants. k can be an integer. k can be different in different implementations. For example, k can be, be about, be at least, be at least about, be at most, or be at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number of a range between any two of these values.

In some embodiments, the plurality of sequence reads is obtained from DNA sequencing, wherein the plurality of sequence reads is obtained from RNA sequencing, wherein the plurality of sequence reads is obtained from bulk RNAseq, and/or wherein the plurality of sequence reads is obtained from single-cell RNAseq. In some embodiments, the sample comprises: a DNA sample, an RNA sample, a bulk RNA sample, a single-cell RNA sample, or a combination thereof. In some embodiments, the plurality of sequence reads comprises a plurality of paired-end sequence reads, and/or wherein the plurality of sequence reads comprises a plurality of single-end sequence reads. In some embodiments, the sample comprises one or more cells, one or more single cells, cell-free DNA (cfDNA), cell-free RNA (cfRNA), or a combination thereof.

In some embodiments, the method comprises: trimming the plurality of sequence reads to generate a plurality of trimmed sequence reads. Determining one or more variants present in the plurality of sequence reads can comprise: determining one or more variants present in the plurality of trimmed sequence reads using the index of the plurality of VCRSs. In some embodiments, the method comprises: filtering the plurality of sequence reads to generate a plurality of filtered sequence reads. Determining one or more variants present in the plurality of sequence reads can comprise: determining one or more variants present in the plurality of filtered sequence reads using the index of the plurality of VCRSs.

In some embodiments, determining the one or more variants present in the plurality of sequence reads comprises: generating a variant count matrix. The variant count matrix can comprise a cell x variant matrix or a sample x variant matrix. Determining the one or more variants present in the plurality of sequence reads can comprise: determining the one or more variants present in the plurality of sequence reads using the variant count matrix. In some embodiments, the method comprises: validating the variant count matrix to generate a validated variant count matrix. Determining the one or more variants present in the plurality of sequence reads can comprise: determining the one or more variants present in the plurality of sequence reads using the validated variant count matrix. In some embodiments, the method comprises: thresholding the variant count matrix to generate a thresholded variant count matrix. Determining the one or more variants present in the plurality of sequence reads can comprises determining the one or more variants present in the plurality of sequence reads using the thresholded variant count matrix.

In some embodiments, determining the one or more variants in the plurality of sequence reads comprises: determining the one or more variants present in the plurality of sequence reads using pseudoalignment. The pseudoalignment can comprise k-mer-based pseudoalignment. The pseudoalignment can comprise kallisto pseudoalignment. In some embodiments, pseudoalignment comprises building a k-mer hash map. The pseudoalignment can comprise creating a de Bruijn graph.

Detecting Mutational Signatures with k-Mer-Based Pseudoalignment

Efficiently describing mutations driving diseases such as cancer, heart disease, and neurological disorders is crucial for precision medicine. Applications include identifying mutations in liquid biopsies, cell type-specific mutation identification, rapidly deploying screening of samples for mutations, and detecting mutations in sequencing data with high sensitivity. However, determining these mutations is challenging due to the vast size of genetic data and inherent biological noise.

In RNA sequencing, a patient's RNA is reverse transcribed to cDNA, sheared into smaller fragments, amplified through polymerase chain reaction (PCR), sequenced to produce a set of ˜150 bp long sequences called reads, and then aligned to a reference genome to determine which gene each read corresponds to. Current methods for detecting mutations in transcriptomic data involve a process called variant calling. Variant calling can detect a mutation if a substantial number of reads possess a nucleotide string that differs from the reference genome. However, due to mistakes with alignment, sequencing errors during reverse transcription or amplification, nucleotide detection errors, and other sources of noise and uncertainty, variant calling requires a large number of reads (on the order of 10 s of reads) to possess a particular variant to detect this variant in the data. Hence, variant calling lacks sensitivity to lowly expressed mutations. Additionally, current algorithms for variant calling are time-consuming and computationally expensive. Therefore, they are not feasible for modern sequencing experiments, which routinely generate terabytes of data. Moreover, existing methods for variant calling lack the ability to track cell barcode assignments and, therefore, are unable to retain the single-cell resolution of single-cell RNA sequencing (RNAseq) data. Finally, variant calling does not make an effort to distinguish pathogenic from benign variants, as it can only describe the occurrence of variants in the data without qualitative metadata. The presently disclosed method addresses all of these issues.

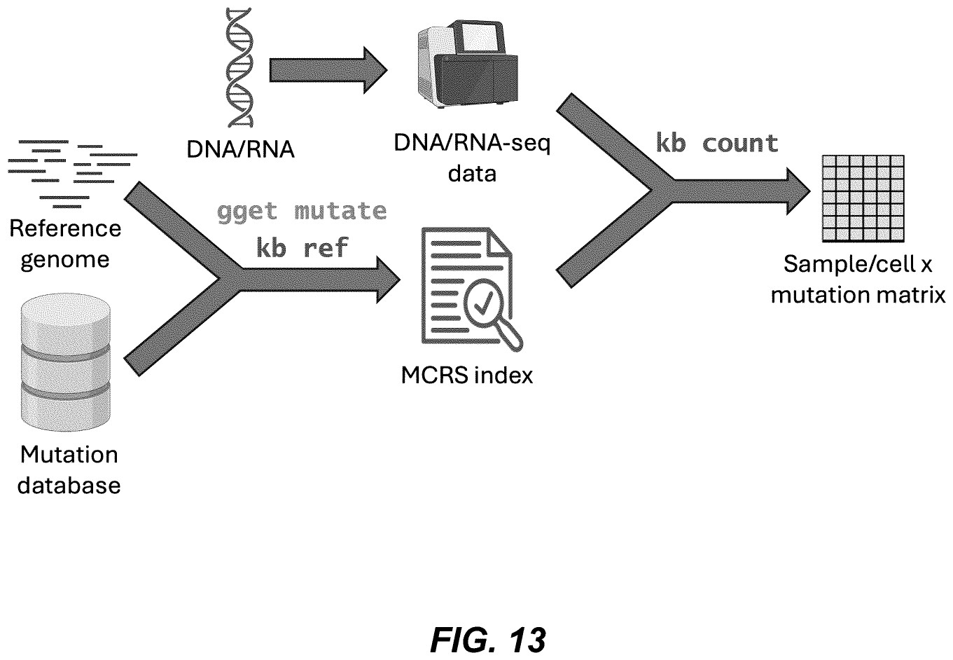

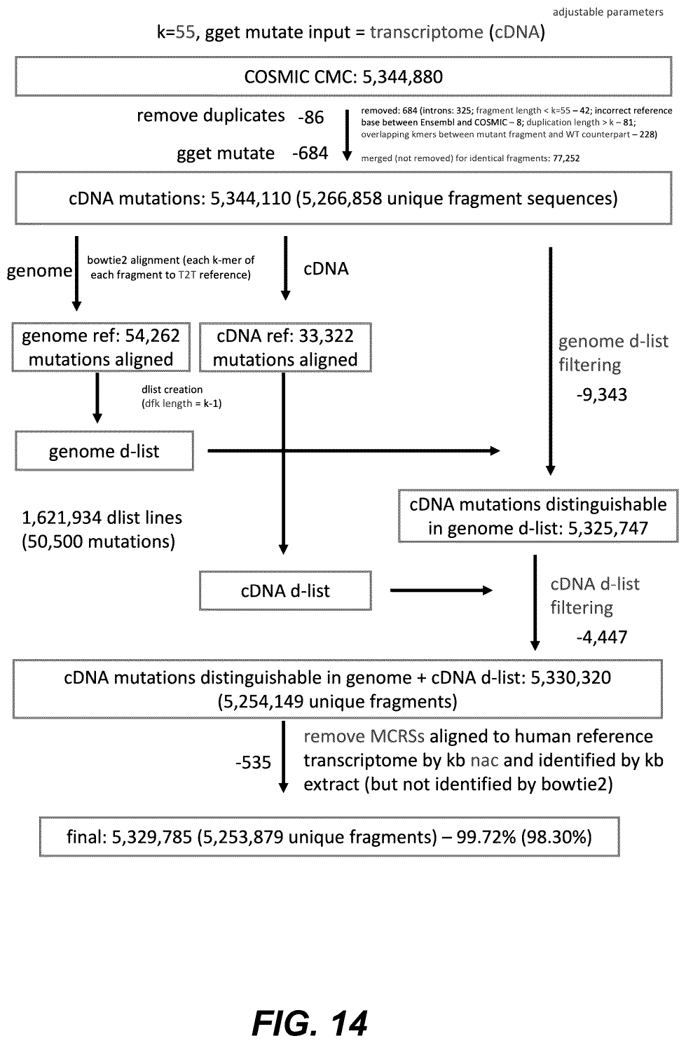

A process was created, by which mutations can be accurately, sensitively, and quickly detected in sequencing reads in bulk or single-cell RNAseq or DNAseq data using a predetermined list of mutations of interest. This process involved: (a) creating a reference index-like data structure for alignment including specific short nucleotide sequences containing each mutation and surrounding information from a database of mutations/variants (FIG. 13, left), (b) aligning RNAseq data to this custom reference using a k-mer-based alignment process (FIG. 13, right), and (c) controlling for false positive mutation detection (i.e., the algorithm detecting a mutation when the mutation was not truly present) (FIG. 14). In summary, the presently disclosed method took DNAseq or RNAseq data, a standard reference genome, and a database of mutations as input and output a sample/cell by mutation occurrence matrix.

To accomplish step (a), a module was written in the software package gget called gget mutate. gget mutate took as input a reference genome/transcriptome and the list of mutations in HGVSG/HGVSC format (i.e., the chromosome/transcript of the mutation, the location range of the mutation, and the post-mutation nucleotide(s)) and output a fasta file of the mutation-containing reference sequences (MCRSs). Mutation-containing reference sequences (MCRSs) are also referred to herein as variant-containing reference sequences (VCRSs). These MCRSs were designed such that all k-mers that comprised them (i) contained at least one mutated nucleotide, and (ii) did not overlap anywhere in the reference genome or transcriptome. The gget mutate module computed MCRSs in less than 10 minutes for the human reference transcriptome and a list of 5,344,800 mutations derived from the Catalog of Somatic Mutations in Cancer (COSMIC) database.

To accomplish step (b), the software package kallisto-bustools (kb) was expanded and applied to align RNAseq data to the reference of MCRSs described in step (a). In particular, kb employs an alignment technique called pseudoalignment, which was significantly faster than true alignment while maintaining comparative accuracy. True alignment uses string similarity comparisons to identify regions of similarity between sequencing reads and the reference genome, calculating alignment scores when considering mismatches, insertions, or deletions. Instead, pseudoalignment breaks up the reads and references into k-mers (fragments of length k) and then performs alignment through a procedure necessitating at least one perfect k-mer match as well as the expectation-maximization algorithm to probabilistically assign reads to their most likely targets in the reference. The requirement of a perfect k-mer match allows pseudoalignment to correctly discern mutant from non-mutant sequences even when only a single nucleotide differs, such as in the case of a substitution mutation. Therefore, mutations can be detected even when only a single read possessing the mutation is present in the data, although the true number of required reads can be slightly higher (in the range of 2-5 reads) in order to account for the errors and uncertainty inherent to the sequencing process. kb provides the additional benefit of supporting almost all modern bulk and single-cell RNAseq methods, including retaining single-cell resolution through cell barcode tracking. Alignment of sequencing data using kb can take on the order of minutes for a standard RNAseq dataset containing hundreds of millions of reads or more.

To accomplish step (c), a variety of techniques were employed within gget mutate, kb, and subsequent processing steps. Within gget mutate, measures to minimize false positives included (i) removing poor quality mutations from the reference mutation database (e.g., the true reference nucleotide differs from the expected reference nucleotide as documented in the reference database), (ii) removing MCRSs containing ‘N’s, (iii) adjusting flank lengths to maximize MCRS length while avoiding any overlapping k-mers between the MCRS and its non-mutated counterpart, (iv) removing mutations that contain overlapping k-mers between the MCRS and its non-mutated counterpart after optimal flank shortening, and (v) merging entries with identical MCRSs. After the fasta-format file of MCRSs was created, any MCRSs that contained a k-mer that was also found in the standard reference genome or transcriptome were removed. In the example of the COSMIC mutation database, 99.72% of mutations were retained after performing all of these measures to control for false positives.

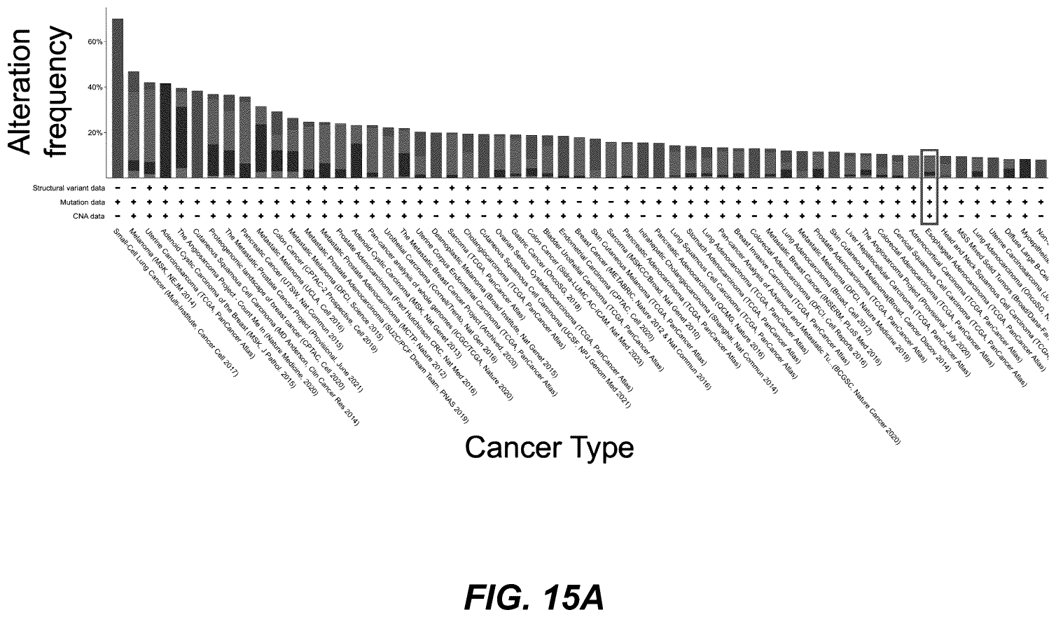

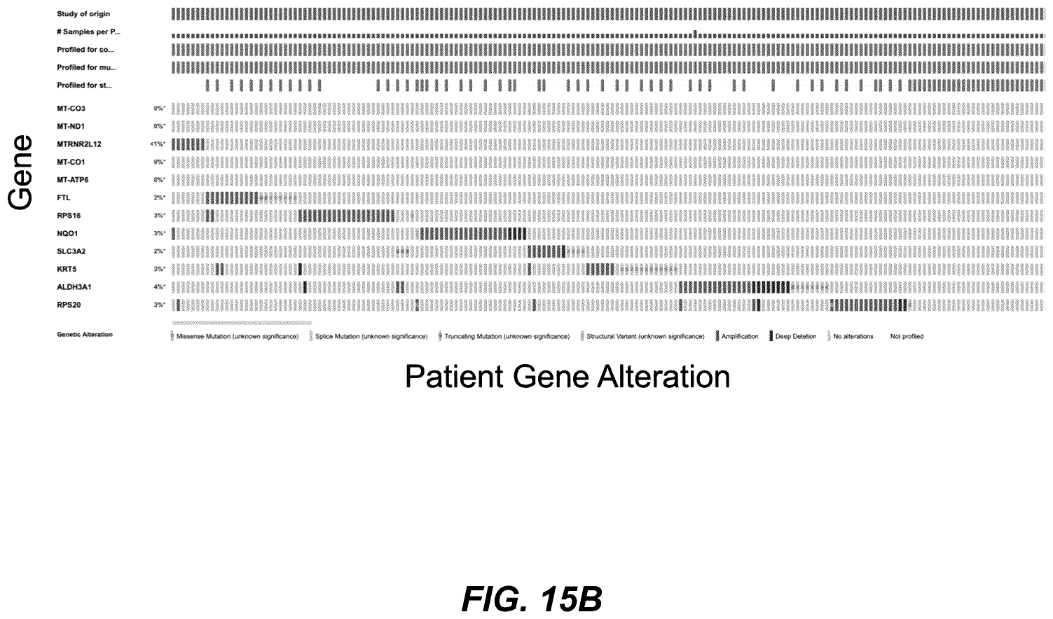

The process of detecting mutational signatures with k-mer-based pseudoalignment was used to analyze bulk RNA-seq data from cancer cell lines in the Cancer Cell Line Encyclopedia, which identified the expected mutations for genes with documented specificity for their respective cancers (FIG. 15A-FIG. 15B). In particular, for the esophageal cancer cell line selected for analysis, the top 10 mutated genes had sensitivity for demonstrating the presence of esophageal cancer (FIG. 15A). When analyzing which genes were most mutated or altered, it was found that the FTL, RPS16, and NQO1 genes were most affected (FIG. 15B). Steps to improve the specificity of this workflow involved optimizing the number of top mutated genes to select, re-applying the pipeline with updates for controlling for false positives, and running processing on the resulting mutation matrix to remove genes or mutations that are not of interest. These results showed promise in the association of the mutation matrix with its respective cancer type.

To make use of the method, the following were involved: (a) obtaining DNAseq or RNAseq data (through experimental/synthetic data generation, obtaining publicly available data, etc.), (b) obtaining or generate an optimized MCRS reference index with a computer (e.g., downloaded from our website or generated using the currently disclosed workflow involving gget mutate and kb, which requires a mutation database in csv/tsv format, e.g., the publicly available database COSMIC, and the standard reference genome compatible with that mutation database, e.g., the publicly available human reference genome from Ensembl), and (c) aligning the sequencing data to the optimized MCRS reference index (e.g., utilizing kb). For the specific implementation of this workflow described herein, the software packages gget version ≥0.28.6 (if creating a custom MCRS reference) and kb-python version ≥0.28.0 (version ≥0.28.2@devel recommended for additional optional functionality) along with their respective software dependencies were used.

A process was created by which one can accurately, sensitively, and quickly detect mutations or variants in sequencing reads in bulk or single-cell RNAseq or DNAseq data using a predetermined list of mutations/variants of interest. This process involved (a) creating a reference index-like data structure for alignment including specific short nucleotide sequences containing each mutation/variant and surrounding information from a database of mutations/variants, (b) aligning RNAseq data to this custom reference using a k-mer-based alignment process, and (c) controlling for false positive mutation/variant detection (i.e., the algorithm detecting a mutation when the mutation was not truly present). The method can take DNAseq or RNAseq data, a standard reference genome, and a database of mutations/variants as input and outputs a sample by mutation/variance or cell by mutation/variance occurrence matrix. While this method can be implemented with the software tools gget (via the gget mutate module) and kallisto-bustools, any tool which can detect mutations/variants from a predefined list in sequencing data and control for false positive hits could accomplish the same task. Variant calling can identify variations by comparing all reads collectively to each other and to the reference genome. The presently disclosed method is an improvement on typical variant calling techniques in that the presently disclosed method (a) had the potential for higher sensitivity of detection mutations/variants, (b) operated on a pre-defined list of mutations/variants that can potentially have met associated metadata, (c) had additional measures to control for false positive hits, (d) supported DNA sequencing, bulk RNA sequencing, and single cell RNA sequencing methods, and (e) was much more computationally efficient.

### Identifying Mutation-Containing or Variant Sequences

Disclosed herein is a method for the identification of mutation-containing or variant sequences in DNA or RNA sequencing reads through. In some embodiments, the method comprises: a) the generation of an optimized reference index from a predetermined list of mutations or variants of interest. The method can comprise: b) alignment of reads from a single-cell or bulk transcriptomics or genomics experiment to the optimized reference index.

In some embodiments, the alignment is performed through pseudo- or quasi-alignment with k-mers. In some embodiments, the reads are obtained using single-cell RNA sequencing and unique molecular identifiers (UMIs) are also tracked. In some embodiments, the reads are obtained using single-cell RNA sequencing and cell barcodes are tracked to retain single-cell resolution.

In some embodiments, the reference index includes nucleotide sequences, each containing a mutation or variant and surrounding genome and/or transcriptome information from a database of mutations or variants (referred to as mutation-containing reference sequences (MCRSs)).

In some embodiments, the sequences in the reference index and/or the reference index are optimized to control for false positive mutation detection. In some embodiments, the sequences in the reference index consist of k-mers that each contain at least one mutated nucleotide. In some embodiments, the sequences in the reference index includes k-mers that are not identical to any k-mers in reference genome(s) for the species of interest. In some embodiments, the sequences in the reference index consist of k-mers that are not identical to any k-mers in reference transcriptome(s) for the species of interest.

In some embodiments, the mutations or variants in the mutation or variant database are interpreted such that conversion between genomic, transcriptomic, or CoDing Sequence reference coordinates and strand information is made possible.

In some embodiments, the sequences in the reference index are optimized by removing poor-quality mutations from the reference mutation database. In some embodiments, the sequences in the reference index are optimized by removing sequences containing non-standard nucleotide characters. In some embodiments, the sequences in the reference index are optimized by removing sequences for which the true reference nucleotide differs from the expected reference nucleotide as documented in the mutation/variant database.

In some embodiments, the sequences in the reference index are adjusted to maximize their length while avoiding any overlapping k-mers between the index sequence (or MCRS) and its non-mutated counterpart. In some embodiments, the sequences in the reference index that contain overlapping k-mers between the index sequence (or MCRS) and its non-mutated counterpart post-optimization are removed.

In some embodiments, identical entries in the reference index are merged. In some embodiments, sequences in the reference index of length <k (where k is a whole number) are removed.

In some embodiments, any sequences in the reference index that contain a k-mer that is also found in reference genome(s) and/or transcriptome(s) of the species of interest post-optimization are masked. In some embodiments, any sequences in the reference index that contain a k-mer that is also found in reference genome(s) and/or transcriptome(s) of the species of interest post-optimization are removed.

In some embodiments, a mutation-containing reference sequence (MCRS) flank-shortening algorithm is used to ensure MCRS dissimilarity from the reference genome(s) and/or transcriptome(s) of the species of interest.

In some embodiments, the method comprises controlling for false positives using true alignment of the sequences in the reference index to a reference genome(s) and/or transcriptome(s) of the species of interest. In some embodiments, the method comprises controlling for false positives using pseudoalignment of the sequences in the reference index to a reference genome(s) and/or transcriptome(s) of the species of interest.

In some embodiments, the method is used for the optimization of the Catalog Of Somatic Mutations In Cancer (COSMIC) database. In some embodiments, the human reference genome is used for the optimization of the COSMIC database. In some embodiments, the human reference transcriptome is used for the optimization of the COSMIC database. In some embodiments, the human reference CoDing Sequences is used for the optimization of the COSMIC database.

### Execution Environment

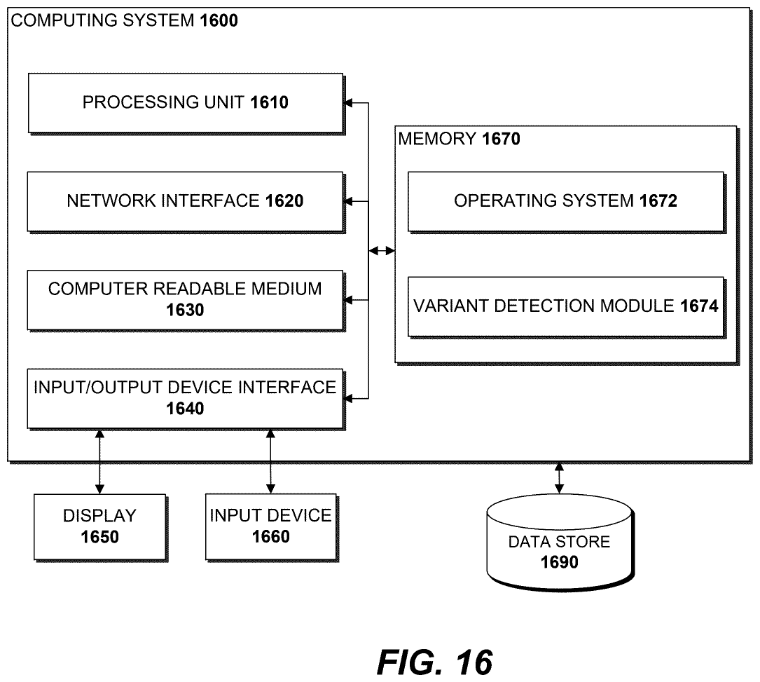

FIG. 16 depicts a general architecture of an example computing device 1600 that can be used in some embodiments to execute the processes and implement the features described herein. The general architecture of the computing device 1600 depicted in FIG. 16 includes an arrangement of computer hardware and software components. The computing device 1600 may include many more (or fewer) elements than those shown in FIG. 16. It is not necessary, however, that all of these generally conventional elements be shown in order to provide an enabling disclosure. As illustrated, the computing device 1600 includes a processing unit 1610, a network interface 1620, a computer readable medium drive 1630, an input/output device interface 1640, a display 1650, and an input device 1660, all of which may communicate with one another by way of a communication bus. The network interface 1620 may provide connectivity to one or more networks or computing systems. The processing unit 1610 may thus receive information and instructions from other computing systems or services via a network. The processing unit 1610 may also communicate to and from memory 1670 and further provide output information for an optional display 1650 via the input/output device interface 1640. The input/output device interface 1640 may also accept input from the optional input device 1660, such as a keyboard, mouse, digital pen, microphone, touch screen, gesture recognition system, voice recognition system, gamepad, accelerometer, gyroscope, or other input device.

The memory 1670 may contain computer program instructions (grouped as modules or components in some embodiments) that the processing unit 1610 executes in order to implement one or more embodiments. The memory 1670 generally includes RAM, ROM and/or other persistent, auxiliary or non-transitory computer-readable media. The memory 1670 may store an operating system 1672 that provides computer program instructions for use by the processing unit 1610 in the general administration and operation of the computing device 1600. The memory 1670 may further include computer program instructions and other information for implementing aspects of the present disclosure.

For example, in one embodiment, the memory 1670 includes a module 1674 for variant detection (such as mutation determination). In addition, memory 1670 may include or communicate with the data store 1690 and/or one or more other data stores that store, for example, inputs, results (e.g., intermediate results or final results), and outputs of variant detection.

### ADDITIONAL CONSIDERATIONS

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1. A method for determining one or more variants:
under control of a hardware processor
receiving variant information of a plurality of variants and an associated reference;
generating a plurality of variant-containing reference sequences (VCRSs) from the variant information of the plurality of variants and an associated reference;
building an index of the plurality of VCRSs;
receiving a plurality of sequence reads obtained from a sample; and
determining one or more variants present in the plurality of sequence reads using the index of the plurality of VCRSs.

2. (canceled)

3. (canceled)

4. The method of claim 1, wherein receiving the variant information of a plurality of variants comprises: receiving the variant information of the plurality of variants from a database of variants, optionally wherein the database of variants comprises Catalog Of Somatic Mutations In Cancer (COSMIC).

5. The method of claim 1, wherein the reference associated with the variant information of the plurality of variants is the reference from which the plurality of variants are annotated.

6. The method of claim 1, wherein the reference comprises a reference genome or a reference transcriptome.

7. The method of claim 1, wherein a VCRS of the plurality of VCRSs comprises a variant region flanked by a plurality of nucleotides (w) flanking each side of the variant region, optionally wherein w is strictly less than k.

8. The method of claim 1, wherein a VCRS of the plurality of VCRSs comprises a variant region flanked by a first plurality of nucleotides (w1) flanking left side of the variant region and a second plurality of nucleotides (w2) flanking right side of the variant region, optionally wherein w1 and/or w2 is strictly less than k.

9. The method of claim 1, comprising: shortening a sequence flank of one or more VCRSs of the plurality of VCRSs.

10. The method of claim 1, comprising trimming one or more VCRSs of the plurality of VCRSs prior to building the index of the plurality of VCRSs, and/or filtering one or more VCRSs of the plurality of VCRSs prior to building the index of the plurality of VCRSs.

11. The method of claim 1, wherein each VCRS corresponds to one of the plurality of variants.

12. The method of claim 1, wherein each k-mer of a VCRS of the plurality of plurality of VCRS comprises at least one variant nucleotide of the corresponding variant of the plurality of variants.

13. The method of claim 1,
wherein the plurality of sequence reads is obtained from DNA sequencing,
wherein the plurality of sequence reads is obtained from RNA sequencing,
wherein the plurality of sequence reads is obtained from bulk RNAseq,
wherein the plurality of sequence reads is obtained from single-cell RNAseq,
wherein the plurality of sequence reads comprises a plurality of paired-end sequence reads, and/or
wherein the plurality of sequence reads comprises a plurality of single-end sequence reads.

14. The method of claim 1, wherein the sample comprises: a DNA sample, an RNA sample, a bulk RNA sample, or a single-cell RNA sample, and/or wherein the sample comprises one or more cells, one or more single cells, cell-free DNA (cfDNA), cell-free RNA (cfRNA), or a combination thereof.

15. (canceled)

16. (canceled)

17. The method of claim 1, comprising: trimming the plurality of sequence reads to generate a plurality of trimmed sequence reads, wherein determining one or more variants present in the plurality of sequence reads comprises: determining one or more variants present in the plurality of trimmed sequence reads using the index of the plurality of VCRSs.

18. The method of claim 1, comprising: filtering the plurality of sequence reads to generate a plurality of filtered sequence reads, wherein determining one or more variants present in the plurality of sequence reads comprises: determining one or more variants present in the plurality of filtered sequence reads using the index of the plurality of VCRSs.

19. The method of claim 1, wherein determining the one or more variants present in the plurality of sequence reads comprises:
generating a variant count matrix, optionally wherein the variant count matrix comprises a cell x variant matrix or a sample x variant matrix; and
determining the one or more variants present in the plurality of sequence reads using the variant count matrix.

20. The method of claim 19, comprising: validating the variant count matrix to generate a validated variant count matrix, wherein determining the one or more variants present in the plurality of sequence reads comprises: determining the one or more variants present in the plurality of sequence reads using the validated variant count matrix.

21. The method of claim 19, comprising: thresholding the variant count matrix to generate a thresholded variant count matrix, wherein determining the one or more variants present in the plurality of sequence reads comprises: determining the one or more variants present in the plurality of sequence reads using the thresholded variant count matrix.

22. The method of claim 1, wherein determining the one or more variants in the plurality of sequence reads comprises: determining the one or more variants present in the plurality of sequence reads using pseudoalignment, optionally wherein the pseudoalignment comprises k-mer-based pseudoalignment, optionally wherein the pseudoalignment comprises kallisto pseudoalignment.

23. The method of claim 22, wherein the pseudoalignment comprises building a k-mer hash map, and/or wherein the pseudoalignment comprises creating a de Bruijn graph.

24. (canceled)

25. (canceled)

26. A system for determining one or more variants comprising:
non-transitory memory configured to store executable instructions and a plurality of variant-containing reference sequences (VCRSs), wherein the plurality of VCRSs is generated from variant information of a plurality of variants and an associated reference; and
a hardware processor in communication with the non-transitory memory, the hardware processor programmed by the executable instructions to perform:
receiving a plurality of sequence reads obtained from a sample; and
determining one or more variants present in the plurality of sequence reads using the plurality of VCRSs.

27.-47. (canceled)


SEQUENCE LISTING (4 sequences)

SEQ ID NO: 1 - DNA, 16 bp, synthetic construct
  source 1..16: mol_type = other DNA
aaaaaaaaaaatttat

SEQ ID NO: 2 - DNA, 29 bp, synthetic construct
  source 1..29: mol_type = other DNA
atcgactcagctagctcagtcagcatcga

SEQ ID NO: 3 - DNA, 22 bp, synthetic construct
  source 1..22: mol_type = other DNA
cgcgcgacatctactcatcgcg

SEQ ID NO: 4 - DNA, 28 bp, synthetic construct
  source 1..28: mol_type = other DNA
catctactcatctactactcatactcag